(19) United States
(12) Patent Application Publication
(10) Pub. No.: US 2022/0183588 A1
(43) Pub. Date: Jun. 16, 2022

(54) GAIT CYCLE DETERMINATION SYSTEM, GAIT CYCLE DETERMINATION METHOD, AND PROGRAM STORAGE MEDIUM

(71) Applicant: NEC Corporation, Minato-ku, Tokyo (JP)

(72) Inventors: Kenichiro FUKUSHI, Tokyo (JP); Chenhui HUANG, Tokyo (JP); Noriyuki TONOUCHI, Tokyo (JP); Kazuki IHARA, Tokyo (JP)

(73) Assignee: NEC Corporation, Minato-ku, Tokyo (JP)

(21) Appl. No.: 17/598,997

(22) PCT Filed: Apr. 5, 2019
(86) PCT No.: PCT/JP2019/015054

§ 371 Date: Sep. 28, 2021

(51) Int. Cl. A61B 5/11; A61B 5/103; A61B 5/00

(52) U.S. Cl. CPC A61B 5/112; A61B 5/742; A61B 5/6807; A61B 5/1038

(57) ABSTRACT

In order to easily and accurately determine a walk cycle, this walk cycle determination system is provided with: a reception unit for receiving sensor data including acceleration and angular velocity acquired by a sensor mounted to footwear; a detection unit which generates time-series data of the orientation angle of at least one foot using the acceleration and angular velocity included in the sensor data, and detects maximal values and minimal values from the time-series data of the orientation angle; and a determination unit for determining the walk cycle based on the sequence of the maximal values and the minimal values.

### TECHNICAL FIELD

The present invention relates to a gait cycle determination device, a gait cycle determination method, and a program for determining a gait cycle.

### BACKGROUND ART

With growing interest in healthcare for physical condition management, a technique has been developed for measuring a gait easily and accurately using sensor data acquired by a sensor attached to a body.

PTL 1 discloses a walk pattern processing device that acquires a two-dimensional pressure distribution based on walking by a pressure sensor and analyzes time series data of the acquired pressure distribution to acquire a walk pattern. The device of PTL 1 creates a superimposed image by superimposing a time-series pressure distribution image in a time direction, extracts a plurality of foot pressure mass regions from the superimposed image, and detects parameters representing characteristics of walking by performing association with the time-series pressure distribution image for each foot pressure cluster region.

PTL 2 discloses a method of measuring acceleration in a left-right direction during walking of a subject at a predetermined measurement cycle using an acceleration sensor attached to the waist of the subject, and detecting the gait cycle of the subject using the measured acceleration in the left-right direction. In the method of PTL 2, a threshold for determining a start of forward movement is set for each of the left and right legs, and a walking state is periodically inspected based on a magnitude relationship between a difference between moving averages of accelerations in the left and right direction at two consecutive time intervals and the threshold set for each of the left and right legs.

PTL 3 discloses a gait cycle detection device that detects a gait cycle of a subject during walking. The device of PTL 3 detects a peak equal to or higher than a threshold from a power spectrum calculated by frequency analysis of acceleration in a vertical direction or a front-back direction during walking of the subject, and detects a gait cycle from a peak frequency corresponding to the detected peak.

PTL 4 discloses a walking speed estimation device that estimates a walking speed using a detection result of an angular velocity sensor attached to a thigh. The device of PTL 4 repeatedly calculates the walking speed at every predetermined time interval, which is a predetermined calculation cycle, based on angular velocity information of the thigh. PTL 4 discloses setting two thresholds in order to discard a characteristic point of an inappropriate angular velocity.

PTL 5 discloses a walking analysis method for detecting an angular velocity according to a motion of a body part of a subject and calculating a gait cycle from the detected angular velocity. In the method of PTL 5, an angular velocity according to a motion of a body part accompanying a stepping motion of a subject is detected, and a gait cycle is calculated based on a stepping cycle calculated based on a variation in the detected angular velocity.

### CITATION LIST

### Patent Literature

[PTL 1] JP 3298793 B2
[PTL 2] JP 2017-074263 A
[PTL 3] JP 2005-342254 A
[PTL 4] JP 2016-214377 A
[PTL 5] JP 2011-250945 A

### SUMMARY OF INVENTION

### Technical Problem

In the method of PTL 1, the gait cycle is determined using a sheet-like foot pressure sensor installed on the floor. Accordingly, the method of PTL 1 has a problem that the device becomes large-scale and the gait cycle can be measured only within the range of the foot pressure sensor.

In the method of PTL 2, focusing on motion of the waist, the threshold is provided for acceleration to detect a transition state of the foot. However, the motion of the waist is greatly different from the motion of the foot portion, and thus the method of PTL 2 has a problem that the gait cycle cannot be accurately detected. In addition, in the method of PTL 2, even if the acceleration sensor is attached to the foot of the subject, it is not possible to acquire data that is easy to analyze, and thus the gait cycle cannot be detected accurately.

In the method of PTL 3, the gait cycle is measured based on the peak of the power spectrum that does not include time information. Thus, the method of PTL 3 has a problem that the time and the gait cycle cannot be associated with each other.

In the method of PTL 4, it is necessary to wear the angular velocity sensor on the thigh using a supporter, but it is troublesome to wear the supporter each time to measure a daily gait cycle. In the method of PTL 4, when the walking speed is estimated using the angular velocity information of the thigh, a characteristic point of the angular velocity in the range between the two preset thresholds is discarded. Therefore, the technique of PTL 4 has a problem that many acquired characteristic points of the angular velocity are discarded when the subject is walking slowly.

In the method of PTL 5, the gait cycle is calculated based on the stepping cycle measured by an angular velocity sensor. Thus, the method of PTL 5 has a problem of determining that the subject is walking even in a situation where he or she is not walking.

In order to solve the above-described problems, an object of the present invention is to provide a gait cycle determination system capable of easily and accurately determining a gait cycle.

### Solution to Problem

A gait cycle determination system according to one aspect of the present invention includes a reception unit that receives sensor data including acceleration and angular velocity acquired by a sensor installed on footwear, a detection unit that generates time series data of an attitude angle of at least one foot by using the acceleration and the angular velocity included in the sensor data, and detects a maximal value and a minimal value from the time series data of the attitude angle, and a determination unit that determines a gait cycle based on an order of the maximal value and the minimal value.

A gait cycle determination method according to one aspect of the present invention includes receiving sensor data including acceleration and angular velocity acquired by a sensor installed on at least one footwear, generating time series data of an attitude angle of at least one foot by using the acceleration and the angular velocity included in the sensor data, detecting a maximal value and a minimal value from the time series data of the attitude angle, and determining a gait cycle based on an order of the maximal value and the minimal value.

A program according to one aspect of the present invention causes a computer to execute a process of receiving sensor data including acceleration and angular velocity acquired by a sensor installed on at least one footwear, a process of generating time series data of an attitude angle of at least one foot by using the acceleration and the angular velocity included in the sensor data, a process of detecting a maximal value and a minimal value from the time series data of the attitude angle, and a process of determining a gait cycle based on an order of the maximal value and the minimal value.

### Advantageous Effects of Invention

According to the present invention, it is possible to provide a gait cycle determination system capable of easily and accurately determining a gait cycle.

### BRIEF DESCRIPTION OF DRAWINGS

FIG. 1 is a block diagram illustrating a configuration of a gait cycle determination system according to a first example embodiment of the present invention.

FIG. 2 is a conceptual diagram illustrating an arrangement example of a data acquisition device of the gait cycle determination system according to the first example embodiment of the present invention.

FIG. 3 is a conceptual diagram for explaining a coordinate system of sensor data acquired by the gait cycle determination system according to the first example embodiment of the present invention.

FIG. 4 is a conceptual diagram for explaining a coordinate system of an attitude angle calculated by the gait cycle determination system according to the first example embodiment of the present invention.

FIG. 5 is a conceptual diagram for explaining a gait cycle determined by the gait cycle determination system according to the first example embodiment of the present invention.

FIG. 6 is a conceptual diagram for explaining a change in a gait phase in a gait cycle determined by the gait cycle determination system according to the first example embodiment of the present invention.

FIG. 7 is a state transition diagram representing transitions of a determination result of the gait cycle by the gait cycle determination system according to the first example embodiment of the present invention.

FIG. 8 is a conceptual diagram illustrating an example in which the data acquisition device of the gait cycle determination system according to the first example embodiment of the present invention is installed on an arch of a foot.

FIG. 9 is a graph illustrating an example of time series data of the attitude angle when the data acquisition device of the gait cycle determination system according to the first example embodiment of the present invention is installed on the arch of the foot.

FIG. 10 is a block diagram illustrating a configuration of the data acquisition device of the gait cycle determination system according to the first example embodiment of the present invention.

FIG. 11 is a block diagram illustrating a configuration of a gait cycle determination device of the gait cycle determination system according to the first example embodiment of the present invention.

FIG. 12 is a flowchart for explaining operation of the gait cycle determination device according to the first example embodiment of the present invention.

FIG. 13 is a flowchart for explaining gait cycle determination processing by a determination unit of the gait cycle determination device according to the first example embodiment of the present invention.

FIG. 14 is a block diagram illustrating a configuration of a gait cycle determination system according to a second example embodiment of the present invention.

FIG. 15 is a conceptual diagram illustrating an example in which a data acquisition device of the gait cycle determination system according to the second example embodiment of the present invention is installed on a heel, a toe, and an instep.

FIG. 16 is a graph illustrating an example of time series data of the attitude angle when the data acquisition device of the gait cycle determination system according to the second example embodiment of the present invention is installed on the heel.

FIG. 17 is a graph illustrating an example of time series data of the attitude angle when the data acquisition device of the gait cycle determination system according to the second example embodiment of the present invention is installed on the toe.

FIG. 18 is a graph illustrating an example of time series data of the attitude angle when the data acquisition device of the gait cycle determination system according to the second example embodiment of the present invention is installed on the instep.

FIG. 19 is a flowchart for explaining operation of a gait cycle determination device according to the second example embodiment of the present invention.

FIG. 20 is a flowchart for explaining exclusion processing by an exclusion unit of the gait cycle determination device according to the second example embodiment of the present invention.

FIG. 21 is a flowchart for explaining gait cycle determination processing by the determination unit of the gait cycle determination device according to the second example embodiment of the present invention.

FIG. 22 is a block diagram illustrating a configuration of a gait cycle determination system according to a third example embodiment of the present invention.

FIG. 23 is a conceptual diagram for explaining a gait cycle determined by the gait cycle determination system according to the third example embodiment of the present invention.

FIG. 24 is a flowchart for explaining operation of a gait cycle determination device according to the third example embodiment of the present invention.

FIG. 25 is a flowchart for explaining gait cycle determination processing by the determination unit of the gait cycle determination device according to the third example embodiment of the present invention.

FIG. 26 is a block diagram illustrating a configuration of a gait cycle determination system according to a fourth example embodiment of the present invention.

FIG. 27 is a conceptual diagram illustrating an example in which a data acquisition device of the gait cycle determination system according to the fourth example embodiment of the present invention is installed under an arch of a foot.

FIG. 28 is a conceptual diagram for explaining a gait cycle determined by the gait cycle determination system according to the fourth example embodiment of the present invention.

FIG. 29 is a block diagram illustrating an example of a hardware configuration for achieving the gait cycle determination device according to each example embodiment of the present invention.

### EXAMPLE EMBODIMENT

Hereinafter, example embodiments of the present invention will be described with reference to the drawings. However, although the example embodiments to be described below are technically preferably limited in order to carry out the present invention, the scope of the invention is not limited to the following. In all the drawings used in the following description of the example embodiment, the same reference numerals are given to similar parts unless there is a particular reason. In the following example embodiments, repeated description of similar configurations and operations may be omitted. The directions of arrows in the drawings illustrate an example, and do not limit the directions of signals between blocks.

### First Example Embodiment

First, a gait cycle determination system according to a first example embodiment of the present invention will be described with reference to the drawings. The gait cycle determination system of the present example embodiment calculates an attitude angle using sensor data acquired by an acceleration sensor and an angular velocity sensor disposed on footwear such as a shoe, and determines a gait cycle based on time series data of the attitude angle. For example, the gait cycle determination system of the present example embodiment calculates the attitude angle by using acceleration data and angular velocity data acquired by an inertial measurement unit (IMU) disposed in a shoe footbed (also referred to as an insole).

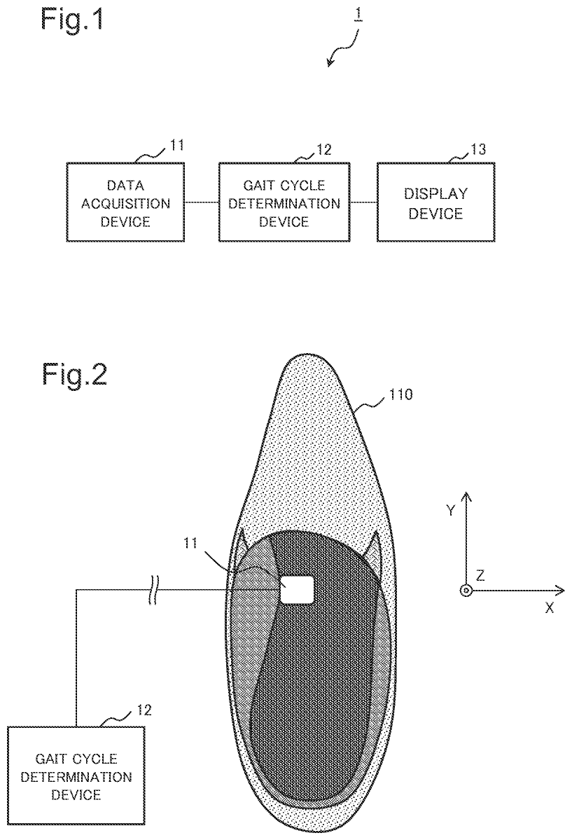

FIG. 1 is a block diagram schematically illustrating a configuration of a gait cycle determination system 1 of the present example embodiment. The gait cycle determination system 1 includes a data acquisition device 11, a gait cycle determination device 12, and a display device 13. The data acquisition device 11 and the gait cycle determination device 12 may be connected by wire or wirelessly. The gait cycle determination device 12 and the display device 13 may be connected by wire or wirelessly, or may be configured as the same terminal device. In a case where a determination result of the gait cycle determination device 12 is not displayed, the display device 13 may be deleted, and the data acquisition device 11 and the gait cycle determination device 12 may constitute the gait cycle determination system 1.

The data acquisition device 11 (also referred to as a sensor) includes at least an acceleration sensor and an angular velocity sensor. The data acquisition device 11 is installed on footwear of the user. The data acquisition device 11 converts data acquired by the acceleration sensor and the angular velocity sensor into digital data (also referred to as sensor data), and transmits sensor data after conversion to the gait cycle determination device 12.

FIG. 2 is a conceptual diagram illustrating an example in which the data acquisition device 11 is installed on a shoe 110. In the example of FIG. 2, the data acquisition device 11 is installed at a position corresponding to a back side of an arch of a foot. The position where the data acquisition device 11 is installed may be a position other than the back side of the arch of the foot as long as the position is inside or on a surface of the shoe 110.

FIG. 3 is a conceptual diagram for explaining a coordinate system of the sensor data acquired by the data acquisition device 11. In the example of FIG. 3, a lateral direction of a walker is set to an X-axis direction (rightward direction is positive), a traveling direction of the walker is set to a Y-axis direction (forward direction is positive), and a gravity direction is set to a Z-axis direction (vertically upward direction is positive).

The data acquisition device 11 is achieved by, for example, an inertial measurement device including an acceleration sensor and an angular velocity sensor. An example of the inertial measurement device is an IMU. The IMU includes a three-axis acceleration sensor and an angular velocity sensor. An example of the inertial measurement device is a vertical gyro (VG). The VG has a configuration similar to that of the IMU, and can output a roll angle and a pitch angle with reference to the gravity direction by a method called strapdown. An example of the inertial measurement device includes an attitude heading reference system (AHRS). The AHRS has a configuration in which an electronic compass is added to the VG. The AHRS can output a yaw angle in addition to the roll angle and the pitch angle. As an example of the inertial measurement device, there is a global positioning system/inertial navigation system (GPS/INS). The GPS/INS has a configuration in which the GPS is added to the AHRS. The GPS/INS can calculate the position in a three-dimensional space in addition to the roll angle, the pitch angle, and the yaw angle, a position can be estimated with high accuracy.

In a case where the acceleration data is used, the attitude angle can be calculated from the magnitude of the acceleration applied in each of the axial directions of the X axis and the Y axis. In a case where the angular velocity data is used, values of the angular velocity with each of the X axis, the Y axis, and the Z axis as central axes can be integrated, so as to calculate the attitude angles around these axes. Meanwhile, high frequency noise changing in various directions is included in the acceleration data, and low frequency noise in the same direction is always included in the angular velocity data. Therefore, by applying a low-pass filter to the acceleration data to remove a high-frequency component, applying a high-pass filter to the angular velocity data to remove a low-frequency component, and combining outputs of the acceleration data and the low-frequency component, accuracy of sensor data from the foot portion on which noise easily rides is improved. It is also possible to improve the accuracy of the sensor data by applying a complementary filter to each of the acceleration data and the angular velocity data and taking a weighted average.

The gait cycle determination device 12 receives the sensor data from the data acquisition device 11. The gait cycle determination device 12 calculates the attitude angle using the received sensor data. In the present example embodiment, the attitude angle is an angle of a sole surface with respect to a horizontal plane (ground). The gait cycle determination device 12 generates time series data of the attitude angle. For example, the gait cycle determination device 12 generates time series data of the attitude angle at predetermined timings or time intervals set in accordance with general gait cycles or gait cycles unique to the user. For example, the gait cycle determination device 12 continues to generate the time series data of the attitude angle during the period in which walking of the user is continued. The timing of generating the time series data of the attitude angles can be set to any timing.

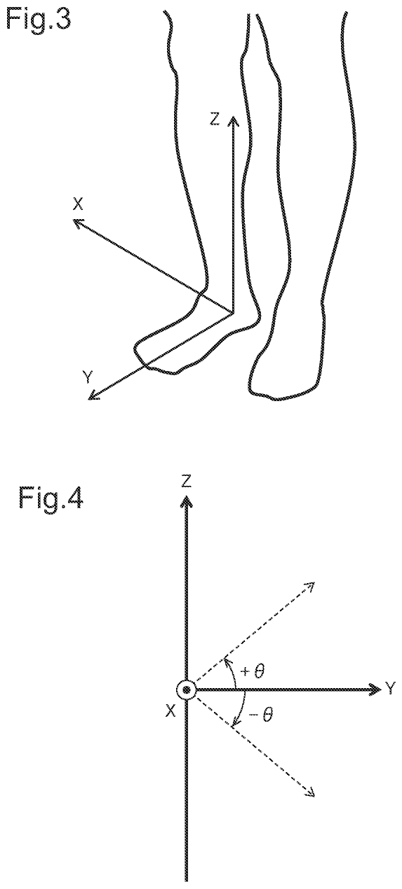

FIG. 4 is a conceptual diagram for explaining a coordinate system of the attitude angle calculated by the gait cycle determination device 12. In FIG. 4, the attitude angle is an angle formed by the ground (positive direction of the Y axis) and the back of the foot (broken line arrow). The gait cycle determination device 12 determines the gait cycle using the attitude angle around the X axis set in the lateral direction of the walker. In the present example embodiment, the attitude angle accompanying an upward rotation around the X axis is positive, and the attitude angle accompanying a downward rotation around the X axis is negative.

The gait cycle determination device 12 detects a maximal value and a minimal value from the time series data of the attitude angle, and determines the gait cycle based on the order of the detected maximal value and minimal value.

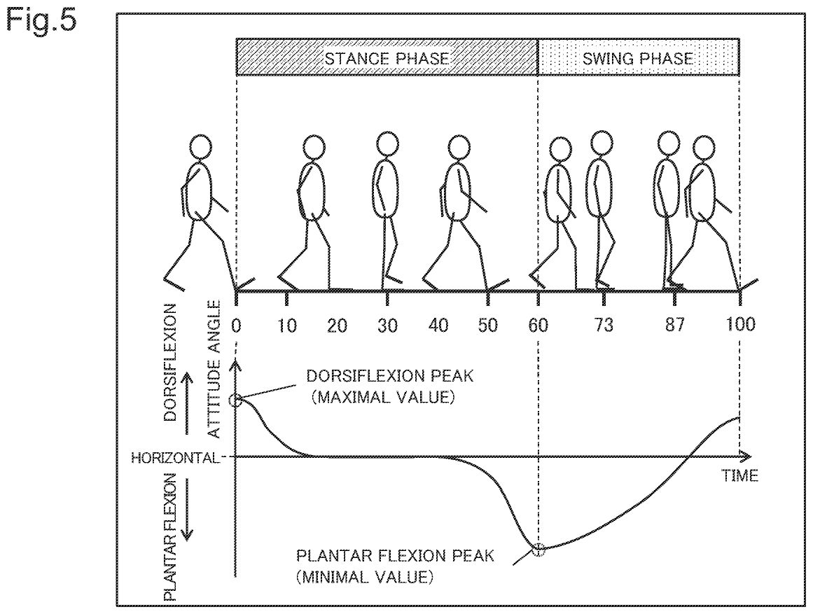

FIG. 5 is a conceptual diagram for explaining a gait cycle determined by the gait cycle determination device 12. The horizontal axis in FIG. 5 represents time normalized with one gait cycle of one leg as 100 percent (also referred to as normalization time). In general, one gait cycle of one foot is roughly divided into a stance phase in which at least a part of the back side of the foot is in contact with the ground and a swing phase in which the back side of the foot is away from the ground. In one gait cycle, the stance phase occupies about 60 percent, and the swing phase occupies about 40 percent.

When the heel of the walker touches the ground (initial grounding), the attitude angle becomes maximal. A peak at which the attitude angle becomes maximal is referred to as a dorsiflexion peak. On the other hand, when the toe of the walker is separated from the ground (toe off), the attitude angle becomes minimal. A peak at which the attitude angle becomes minimal is called a plantar flexion peak. The maximal and minimal of the attitude angle are exchanged when the positive and negative of the attitude angle become opposite depending on the attachment method of the data acquisition device 11.

The gait cycle determination device 12 detects a time when the attitude angle becomes maximal as a start time of the stance phase, and detects a time when the attitude angle becomes minimal as a start time of the swing phase. In other words, the gait cycle determination device 12 detects the time when the attitude angle becomes maximal as an end time of the swing phase, and detects the time when the attitude angle becomes minimal as an end time of the stance phase. The gait cycle determination device 12 determines the gait cycle based on the order relationship between the dorsiflexion peak at which the attitude angle becomes maximal and the plantar flexion peak at which the attitude angle becomes minimal. The gait cycle determination device 12 determines a period from the dorsiflexion peak (maximal) to the next plantar flexion peak (minimal) as the stance phase, and a period from the plantar flexion peak (minimal) to the next dorsiflexion peak (maximal) as the swing phase. That is, in a case where the minimal value is detected after the maximal value, the gait cycle determination device 12 determines that a transition from the stance phase to the swing phase has occurred. On the other hand, in a case where the maximal value is detected after the minimal value, the gait cycle determination device 12 determines that a transition from the swing phase to the stance phase has occurred.

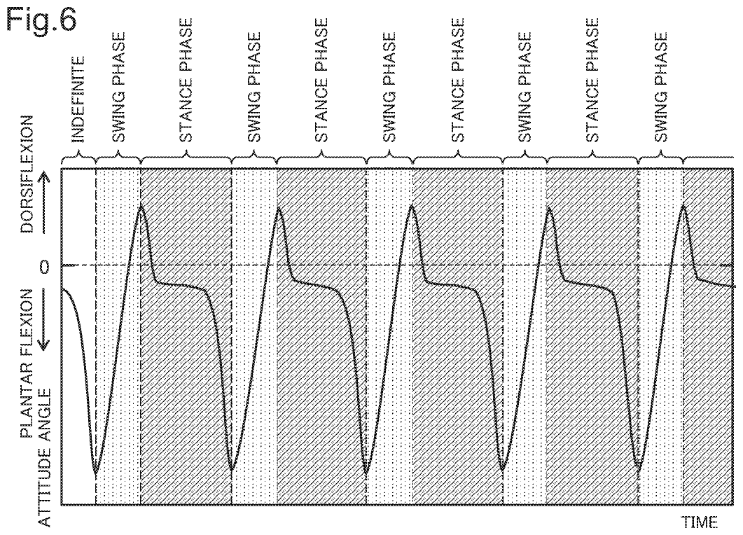

FIG. 6 is a conceptual diagram illustrating an example in which the gait cycle determination device 12 periodically detects the swing phase and the stance phase after detecting walking of the walker. In FIG. 6, the gait phase is indefinite in a period until the attitude angle reaches the minimal first. The gait cycle determination device 12 detects a time when the attitude angle becomes minimal as a start time of the swing phase. The gait cycle determination device 12 detects a time when the attitude angle becomes maximal after the attitude angle becomes minimal as a start time of the stance phase. Then, upon detecting the time when the attitude angle becomes minimal as a start time of the swing phase after the attitude angle becomes maximal, the gait cycle determination device 12 determines that walking of one gait cycle is performed. In a case where the maximal of the attitude angle is detected for the first time, the order of the stance phase and the swing phase is switched.

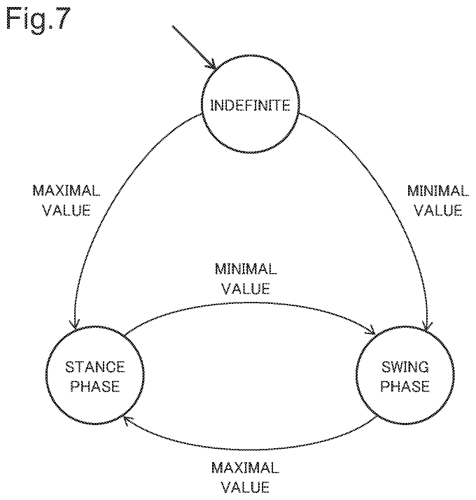

FIG. 7 is a state transition diagram representing transitions of a determination result of the gait cycle. First, the gait cycle determination device 12 detects a minimal value or a maximal value of the attitude angle in an indefinite state. The gait cycle determination device 12 determines that the swing phase is started when the minimal value of the attitude angle is detected, and determines that the stance phase is started when the maximal value of the attitude angle is detected. The gait cycle determination device 12 determines that the stance phase is started when the maximal value is detected in the swing phase, and determines that the swing phase is started when the minimal value is detected in the stance phase. The gait cycle determination device 12 determines the gait cycle by alternately detecting the maximal value of the attitude angle (stance phase) and the minimal value of the attitude angle (swing phase). In a case where the maximal value (stance phase) of the attitude angle and the minimal value (swing phase) of the attitude angle are not alternately detected, the gait cycle determination device 12 determines that the gait cycle is stopped.

The gait cycle determination device 12 outputs the determination result of the gait cycle to the display device 13. For example, the gait cycle determination device 12 outputs the current gait phase (stance phase or swing phase) as a determination result. In addition, for example, the gait cycle determination device 12 may output the ratio of respective durations of the stance phase and the swing phase, a stride, a walking speed, a sensor height, and/or the like as the determination result. An output destination of the determination result of the gait cycle may be, instead of the display device 13, a system or device that measures the number of steps or a gait based on the determination result of the gait cycle. The output destination of the determination result of the gait cycle is not limited to the system or device that measures the number of steps or the gait as long as it is a system or device that uses the determination result.

The gait cycle determination device 12 is achieved by, for example, software (application) or a circuit installed in a portable terminal device such as a smartphone, a mobile phone, a tablet, or a notebook personal computer. In a case where it is used for data analysis of research or the like, for example, the gait cycle determination device 12 may be achieved by software or a circuit installed in an information processing device such as a stationary computer or a server.

The display device 13 acquires the determination result of the gait cycle from the gait cycle determination device 12. The display device 13 displays the acquired determination result on the monitor of the display device 13. For example, the display device 13 causes the monitor to display the gait cycle, the gait phase at the current time, the ratio of respective durations of the stance phase and the swing phase, the walking speed, the stride, height information of the sensor, and/or the like. For example, the ratio of the duration of each of the stance phase and the swing phase is correlated with walking ability, and the ratio of the duration of the swing phase to that of the stance phase becomes smaller in older people. The walking speed, the stride, the height information of the sensor, and the like are related to a health condition, and if the health condition is poor, the walking speed becomes slow, the stride becomes small, and the height of the sensor becomes low. The user viewing the monitor of the display device 13 can estimate the health condition or the like by information displayed on the monitor.

Here, time series data of the attitude angle obtained when the data acquisition device 11 is installed on the arch of the foot will be described with reference to the drawings.

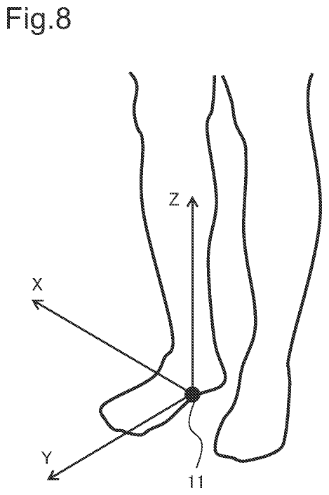

FIG. 8 is a conceptual diagram illustrating an example in which the data acquisition device 11 is installed on the arch of the foot. In the example of FIG. 8, the lateral direction of the walker is set to the X-axis direction (rightward direction is positive), the traveling direction of the walker is set to the Y-axis direction (forward direction is positive), and the gravity direction is set to the Z-axis direction (vertically upward direction is positive).

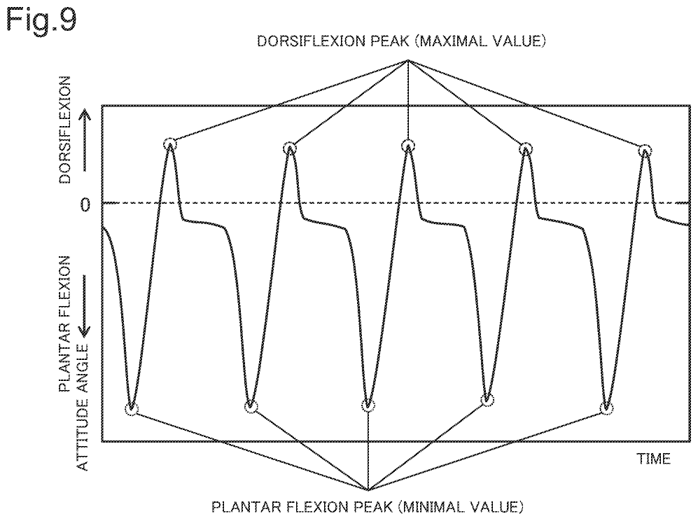

FIG. 9 is a conceptual diagram illustrating an example of the time series data of the attitude angle obtained when the data acquisition device 11 is installed on the arch of the foot. In the example of FIG. 9, after the minimal value (plantar flexion peak) of the attitude angle is detected, the maximal value (dorsiflexion peak) and the minimal value (plantar flexion peak) are alternately detected. A temporal change of the attitude angle becomes once gentle after the maximal value (the dorsiflexion peak) is detected, and then becomes large again. A period in which the temporal change of the attitude angle becomes gentle is a stage in which the foot on the opposite side is separated from the ground, and the body of the walker is supported with one foot. In other words, the period in which the temporal change of the attitude angle becomes gentle is a period from a middle of a mid-stance period to a middle of a terminal stance period. In the example of FIG. 9, in a case where the data acquisition device 11 is installed on the arch of the foot, the maximal value is not detected in a period in which the temporal change of the attitude angle becomes gentle. Therefore, in a case where the data acquisition device 11 is installed on the arch of the foot, the gait cycle can be analyzed using the maximal value and the minimal value detected from the time series data of the attitude angle as they are.

The outline of the configuration of the gait cycle determination device 12 has been described above. Note that the configuration of FIG. 1 is an example, and the configuration of the gait cycle determination device 12 of the present example embodiment is not limited to the mode as it is.

[Data Acquisition Device]

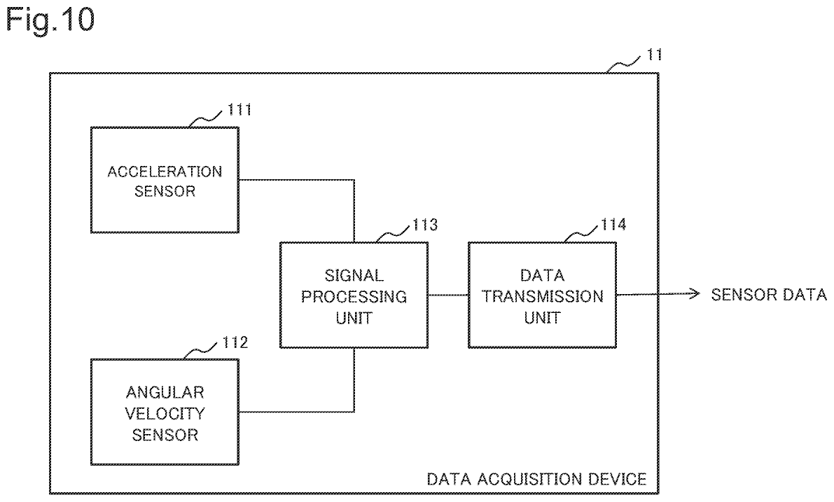

Next, the data acquisition device 11 included in the gait cycle determination system 1 will be described with reference to the drawings. FIG. 10 is a block diagram illustrating an example of a configuration of the data acquisition device 11. The data acquisition device 11 includes an acceleration sensor 111, an angular velocity sensor 112, a signal processing unit 113, and a data transmission unit 114.

The acceleration sensor 111 is a sensor that measures acceleration in three axial directions. The acceleration sensor 111 outputs the measured acceleration to the signal processing unit 113.

The angular velocity sensor 112 is a sensor that measures an angular velocity. The angular velocity sensor 112 outputs the measured angular velocity to the signal processing unit 113.

The signal processing unit 113 acquires each of the acceleration and the angular velocity from each of the acceleration sensor 111 and the angular velocity sensor 112. The signal processing unit 113 converts the acquired acceleration and angular velocity into digital data, and outputs the digital data (sensor data) after conversion to the data transmission unit 114. The sensor data includes at least acceleration data obtained by converting acceleration of analog data into digital data and angular velocity data obtained by converting angular velocity of analog data into digital data. The sensor data may include an acquisition time of raw data of acceleration and angular velocity. The signal processing unit 113 may be configured to output sensor data obtained by performing correction such as mounting error or temperature correction, linearity correction, and/or the like on the acquired raw data of acceleration and angular velocity.

The data transmission unit 114 acquires the sensor data from the signal processing unit 113. The data transmission unit 114 transmits the acquired sensor data to the gait cycle determination device 12. The data transmission unit 114 may transmit the sensor data to the gait cycle determination device 12 via a wire such as a cable, or may transmit the sensor data to the gait cycle determination device 12 via wireless communication. For example, the data transmission unit 114 can be configured to transmit sensor data to the gait cycle determination device 12 via a wireless communication function (not illustrated) conforming to a standard such as Bluetooth (registered trademark) or WiFi (registered trademark).

The example of the configuration of the data acquisition device 11 has been described above. Note that the configuration of FIG. 10 is an example, and the configuration of the data acquisition device 11 included in the gait cycle determination system 1 of the present example embodiment is not limited to the mode as it is.

[Gait Cycle Determination Device]

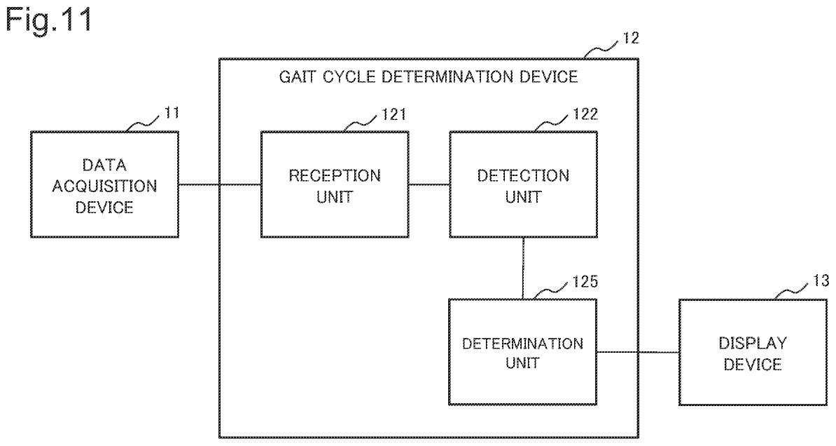

Next, the gait cycle determination device 12 included in the gait cycle determination system 1 will be described with reference to the drawings. FIG. 11 is a block diagram illustrating an example of a configuration of the gait cycle determination device 12. The gait cycle determination device 12 includes a reception unit 121, a detection unit 122, and a determination unit 125.

The reception unit 121 receives sensor data from the data acquisition device 11. The reception unit 121 outputs the acceleration data and the angular velocity data included in the sensor data to the detection unit 122.

The detection unit 122 acquires the acceleration data and the angular velocity data from the reception unit 121. The detection unit 122 calculates the attitude angle using the acquired acceleration data and angular velocity data, and generates time series data of the attitude angle. For example, the detection unit 122 generates time series data of the attitude angle from the acceleration data and the angular velocity data using general-purpose software.

The detection unit 122 detects a maximal value and a minimal value from the time series data of the attitude angle. Upon detecting the maximal value from the time series data of the attitude angle, the detection unit 122 outputs the detected maximal value to the determination unit 125 in association with the acquisition time. Upon detecting the minimal value from the time series data of the attitude angle, the detection unit 122 outputs the detected minimal value to the determination unit 125 in association with the acquisition time.

The determination unit 125 acquires the minimal value or the maximal value from the detection unit 122. The determination unit 125 performs walking determination based on the order of acquiring the minimal value and the maximal value. In a case where the minimal value is acquired after acquiring the maximal value, the determination unit 125 determines that a transition from the stance phase to the swing phase has occurred. In a case where the maximal value is acquired after acquiring the minimal value, the determination unit 125 determines that a transition from the swing phase to the stance phase has occurred. The determination unit 125 outputs the determination result such as the gait phase at the current time to the display device 13. In a case of a configuration not including the display device 13, the determination unit 125 outputs the determination result to a system or a device that is not illustrated.

The example of the configuration of the gait cycle determination device 12 has been described above. Note that the configuration of FIG. 11 is an example, and the configuration of the gait cycle determination device 12 included in the gait cycle determination system 10 of the present example embodiment is not limited to the mode as it is.

(Operation)

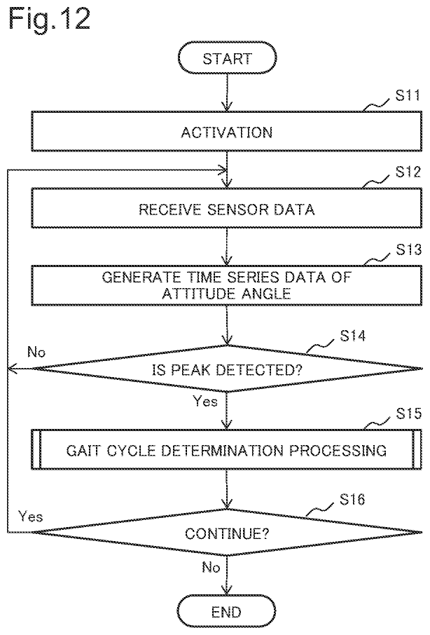

Next, operation of the gait cycle determination device 12 of the present example embodiment will be described with reference to the drawings. FIG. 12 is a flowchart for explaining the operation of the gait cycle determination device 12.

In FIG. 12, first, the gait cycle determination device 12 is activated (step S11).

Next, the gait cycle determination device 12 receives sensor data (acceleration data and angular velocity data) from the data acquisition device 11 (step S12).

Next, the gait cycle determination device 12 calculates the attitude angle using acceleration data and angular velocity data included in the received sensor data, and generates time series data of the attitude angle (step S13).

Then, in a case where a peak is detected from the time series data of the attitude angle generated this time (Yes in step S14), the gait cycle determination device 12 executes the gait cycle determination processing (step S15) using the time series data of the attitude angle and outputs the determination result to the display device 13. In the gait cycle determination processing (step S15), the gait cycle determination device 12 determines the gait cycle based on the order of the maximal peak and the minimal peak. On the other hand, in a case where no peak is detected from the time series data of the attitude angle generated this time (No in step S14), the processing returns to step S12.

After step S15, in a case where the processing is continued (Yes in step S16), the processing returns to step S12. In a case where the processing is ended (No in step S16), the processing according to the flowchart of FIG. 12 is ended.

The example of the operation of the gait cycle determination device 12 has been described above. Note that the flowchart of FIG. 12 is an example, and the operation of the gait cycle determination device 12 of the present example embodiment is not limited to the procedure as it is.

[Gait Cycle Determination Processing]

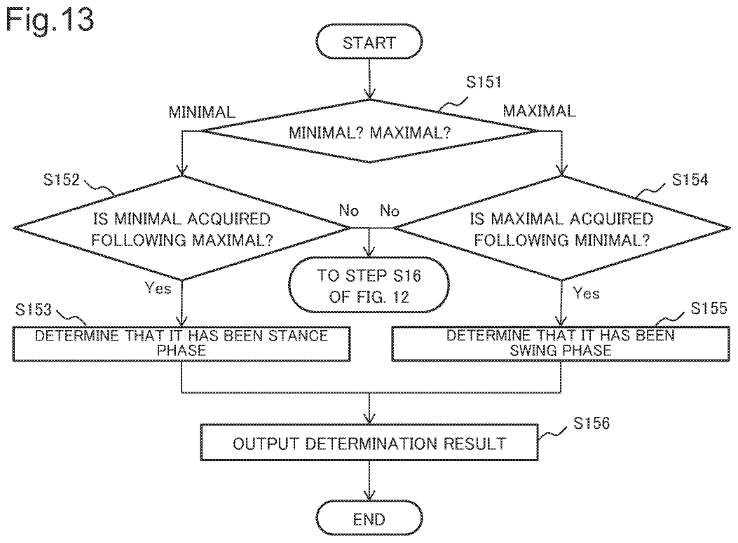

Next, the gait cycle determination processing by the determination unit 125 of the gait cycle determination device 12 of the present example embodiment will be described with reference to the drawings. FIG. 13 is a flowchart for explaining gait cycle determination processing by the determination unit 125.

In FIG. 13, in a case where a minimal peak is acquired from the detection unit 122 (minimal in step S151), the determination unit 125 determines whether the minimal peak is acquired following the maximal peak (step S152). In a case where the minimal peak is acquired following the maximal peak (Yes in step S152), the determination unit 125 determines that the period before the minimal peak has been the stance phase (step S153) and outputs a determination result (step S156). In step S156, the determination unit 125 may output a determination result that the period before the minimal peak has been the stance phase, or may output a determination result that it is the swing phase at the current time. After step S156, the processing proceeds to step S16 in the flowchart of FIG. 12.

On the other hand, in a case where the minimal peak is not acquired following the maximal peak (No in step S152), the processing proceeds to step S16 of the flowchart of FIG. 12. The case where the minimal peak is not acquired following the maximal peak is a case where the peak is not acquired at a predetermined timing or during a predetermined period. For example, in the case where the minimal peak is not acquired following the maximal peak, a determination result that an abnormality is detected in the gait cycle may be output.

In FIG. 13, in a case where the maximal peak is acquired from detection unit 122 (maximal in step S151), the determination unit 125 determines whether the maximal peak is acquired following the minimal peak (step S154). In a case where the maximal peak is acquired following the minimal peak (Yes in step S154), the determination unit 125 determines that the period before the maximal peak has been the swing phase (step S155), and outputs a determination result (step S156). In step S156, the determination unit 125 may output a determination result that the period before the maximal peak has been the swing phase, or may output a determination result that it is the stance phase at the current time. After step S156, the processing proceeds to step S16 in the flowchart of FIG. 12.

On the other hand, in a case where the maximal peak is not acquired following the minimal peak (No in step S154), the processing proceeds to step S16 of the flowchart of FIG. 12. The case where the maximal peak is not acquired following the minimal peak is a case where the peak is not acquired at a predetermined timing or during a predetermined period. For example, in the case where the maximal peak is not acquired following the minimal peak, a determination result that an abnormality is detected in the gait cycle may be output.

The gait cycle determination processing by the determination unit 125 has been described above. Note that the flowchart of FIG. 13 is an example, and the gait cycle determination processing by the determination unit 125 of the present example embodiment is not limited to the procedure as it is.

As described above, the gait cycle determination system of the present example embodiment includes a reception unit, a detection unit, and a determination unit. The reception unit receives sensor data including acceleration and angular velocity acquired by a sensor installed on footwear. The detection unit generates time series data of an attitude angle of at least one foot by using the acceleration and the angular velocity included in the sensor data, and detects a maximal value and a minimal value from the time series data of the attitude angle. The determination unit determines a gait cycle based on an order of the maximal value and the minimal value. As one aspect of the present example embodiment, the determination unit determines a gait phase in a period from a detection time of the maximal value to a detection time of the minimal value that is next as a stance phase, and determines the gait phase in a period from a detection time of the minimal value to a detection time of the maximal value that is next as a swing phase.

As one aspect of the present example embodiment, the gait cycle determination system includes a data acquisition device that is installed on footwear, detects the acceleration and the angular velocity, generates the sensor data including the acceleration and the angular velocity that are detected, and transmits the sensor data that is generated to the reception unit.

As one aspect of the present example embodiment, the gait cycle determination system includes a display device that acquires a determination result by the determination unit and displays the acquired determination result.

The gait cycle determination system of the present example embodiment generates time series data of the attitude angle by using the sensor data acquired by an acceleration sensor and an angular velocity sensor attached to footwear. The gait cycle determination system of the present example embodiment determines the gait cycle based on the maximal value and the minimal value detected from the time series data of the attitude angle. The gait cycle determination system of the present example embodiment determines a detection time of a maximal value as a start time of a stance phase, and determines a detection time of a minimal value as a start time of a swing period. That is, the gait cycle determination system of the present example embodiment determines a gait phase between the detection time of the maximal value and the detection time of the minimal value as the stance phase, and determines a gait phase between the detection time of the minimal value and the detection time of the maximal value as the swing phase.

The gait cycle determination system of the present example embodiment can determine the gait cycle in association with the time by using sensor data acquired by a sensor attached to a foot portion, and thus can accurately determine the gait cycle. That is, with the gait cycle determination system of the present example embodiment, the gait cycle can be easily and accurately determined using the sensor data acquired by the sensor attached to footwear.

### Second Example Embodiment

Next, a gait cycle determination system according to a second example embodiment of the present invention will be described with reference to the drawings. The gait cycle determination system of the present example embodiment is different from that of the first example embodiment in that an exclusion range for excluding the maximal of the attitude angle appearing at a time other than the start time of the stance phase and the minimal of the attitude angle appearing at a time other than the start time of the swing phase is set for the attitude angle.

(Configuration)

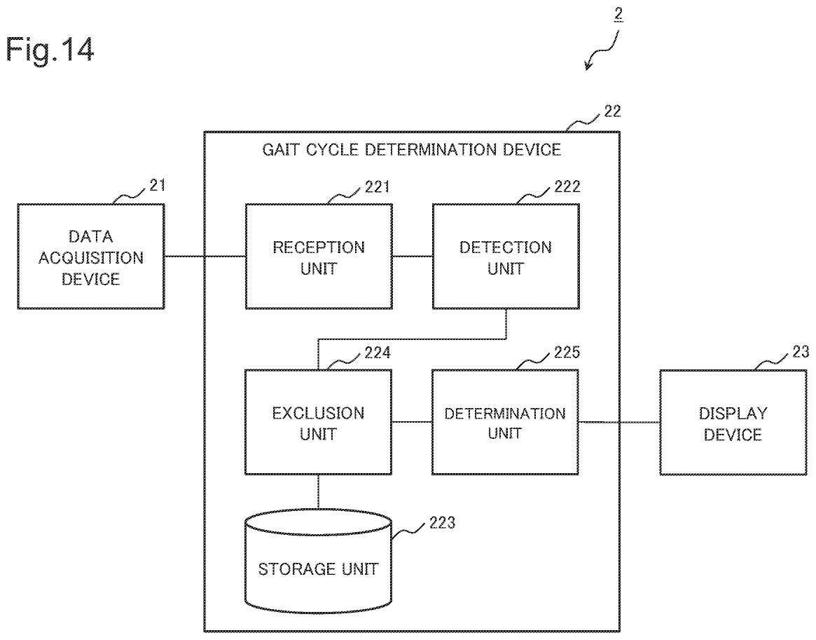

FIG. 14 is a block diagram schematically illustrating a configuration of a gait cycle determination system 2 of the present example embodiment. The gait cycle determination system 2 includes a data acquisition device 21, a gait cycle determination device 22, and a display device 23. The data acquisition device 21 and the gait cycle determination device 22 may be connected by wire or wirelessly. The gait cycle determination device 22 and the display device 23 may be connected by wire or wirelessly, or may be configured as the same terminal device. In a case where a determination result of the gait cycle determination device 22 is not displayed, the display device 23 may be deleted, and the data acquisition device 21 and the gait cycle determination device 22 may constitute the gait cycle determination system 2. Hereinafter, the data acquisition device 21 and the display device 23 are similar in configuration and function to the data acquisition device 11 and the display device 13, respectively, of the first example embodiment, and thus a detailed description thereof will be omitted.

As illustrated in FIG. 14, the gait cycle determination device 22 includes a reception unit 221, a detection unit 222, a storage unit 223, an exclusion unit 224, and a determination unit 225.

The reception unit 221 receives sensor data from the data acquisition device 21. The reception unit 221 outputs acceleration data and angular velocity data included in the sensor data to the detection unit 222.

The detection unit 222 acquires the acceleration data and the angular velocity data from the reception unit 221. The detection unit 222 calculates the attitude angle using the acquired acceleration data and angular velocity data, and generates time series data of the attitude angle. The detection unit 222 detects a maximal value or a minimal value from the time series data of the attitude angle. Upon detecting the maximal value from the time series data of the attitude angle, the detection unit 222 outputs the detected maximal value to the exclusion unit 224. Upon detecting the minimal value from the time series data of the attitude angle, the detection unit 222 outputs the detected minimal value to the exclusion unit 224. The maximal value and the minimal value output from the detection unit 222 include respective values of the maximal value and the minimal value and respective times when the maximal value and the minimal value is detected.

The storage unit 223 (also referred to as a first storage unit) stores thresholds for excluding unnecessary maximal and minimal appearing in the time series data of the attitude angles. Specifically, the storage unit 223 stores a first predetermined value for setting an exclusion upper limit and a second predetermined value for setting an exclusion lower limit. The maximal value detected from the time series data of the attitude angle and the minimal value detected from the time series data of the attitude angle are accumulated in the storage unit 223. The storage unit 223 may be configured to store the maximum value among the maximal values detected from the time series data of the attitude angles and the minimum value among the minimal values detected from the time series data of the attitude angle. The storage unit 223 may be configured to store time series data of the attitude angle.

The exclusion unit 224 acquires one of the maximal value and the minimal value from the detection unit 222. The exclusion unit 224 sets an exclusion range of the attitude angle based on the maximal value and the minimal value acquired from the detection unit 222. The exclusion unit 224 excludes maximal and minimal included in the set exclusion range.

Upon receiving the maximal value, the exclusion unit 224 compares the received maximal value with the maximal value stored in the storage unit 223. The exclusion unit 224 sets a value obtained by subtracting the first predetermined value from the maximum value among maximal values received so far as the exclusion upper limit. In a case where the received maximal value is more than the exclusion upper limit, the exclusion unit 224 outputs the maximal value to the determination unit 225. On the other hand, in a case where the received maximal value is equal to or less than the exclusion upper limit, the exclusion unit 224 does not output the maximal value.

Upon receiving the minimal value, the exclusion unit 224 compares the received minimal value with the minimal value stored in the storage unit 223. The exclusion unit 224 sets, as the exclusion lower limit value, a value obtained by adding the second predetermined value to the minimum value among the minimal values received so far. In a case where the received minimal value is less than the exclusion lower limit, the exclusion unit 224 outputs the minimal value to the determination unit 225. On the other hand, in a case where the received minimal value is equal to or more than the exclusion lower limit, the exclusion unit 224 does not output the minimal value.

The determination unit 225 acquires the minimal value or the minimal value from the exclusion unit 224. The determination unit 225 performs walking determination based on the order of acquiring the minimal value and the maximal value. In a case where the minimal value is acquired after acquiring the maximal value, the determination unit 225 determines that a transition from the stance phase to the swing phase has occurred. In a case where the maximal value is acquired after acquiring the minimal value, the determination unit 225 determines that a transition from the swing phase to the stance phase has occurred. The determination unit 225 outputs the determination result such as the gait phase at the current time to the display device 23. In a case of a configuration not including the display device 23, the determination unit 225 outputs the determination result to a system or a device that is not illustrated.

The example of the configuration of the gait cycle determination device 22 has been described above. Note that the configuration of FIG. 14 is an example, and the configuration of the gait cycle determination device 22 included in the gait cycle determination system 2 of the present example embodiment is not limited to the mode as it is.

Here, time series data of the attitude angle obtained when the data acquisition device 21 is installed on a heel, a toe, an instep, or the like will be described with reference to the drawings.

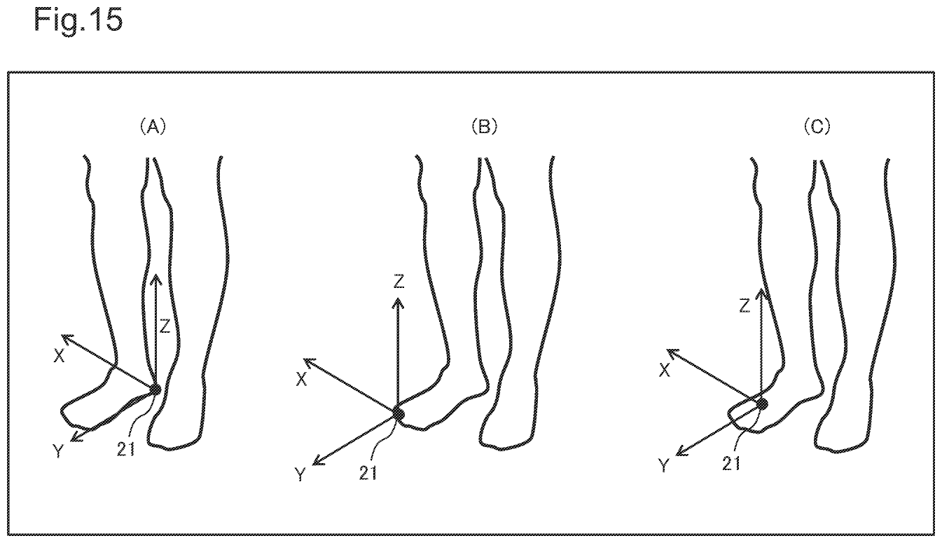

FIG. 15 is a conceptual diagram illustrating an example in which the data acquisition device 21 is installed on a heel (A), a toe (B), and an instep (C). In the example of FIG. 15, the lateral direction of the walker is set to the X-axis direction (rightward direction is positive), the traveling direction of the walker is set to the Y-axis direction (forward direction is positive), and the gravity direction is set to the Z-axis direction (vertically upward direction is positive).

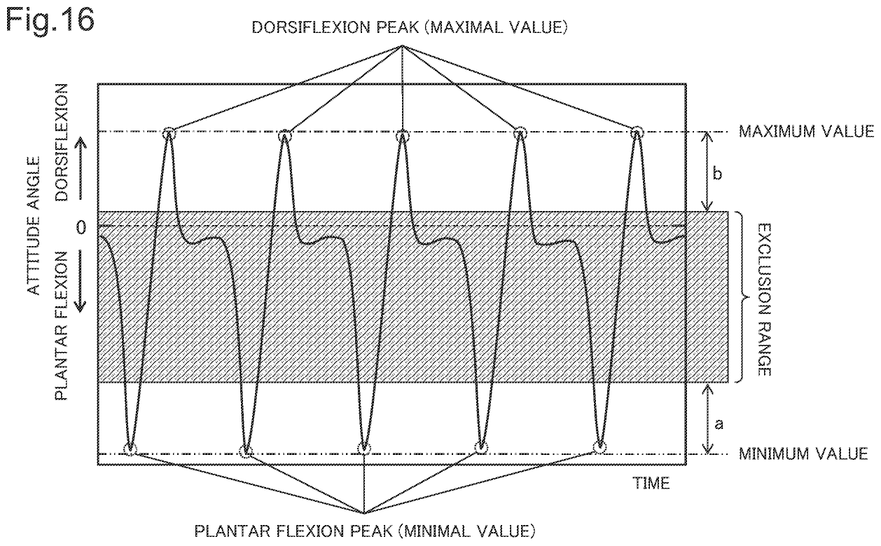

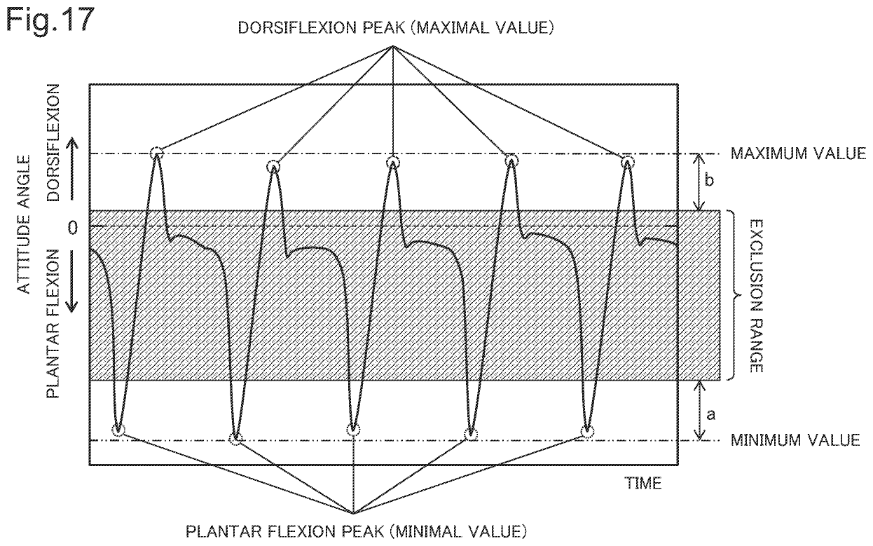

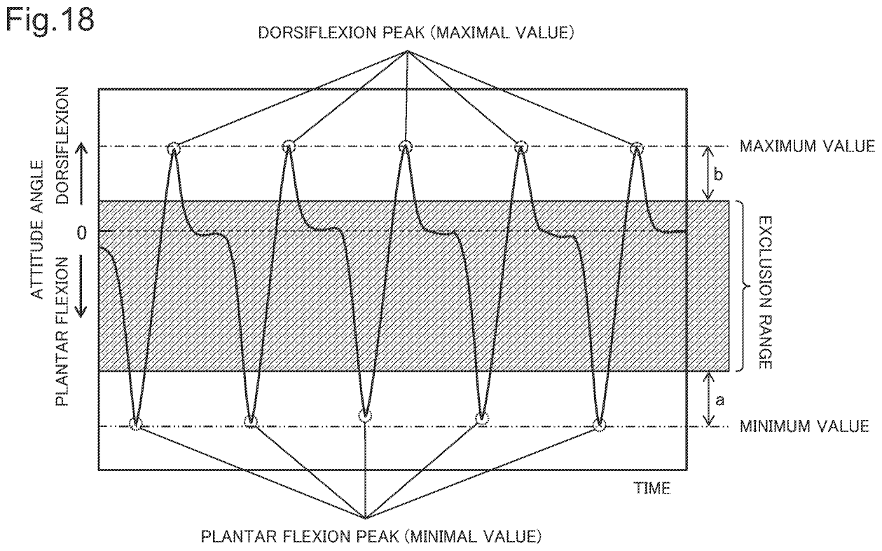

FIG. 16 is a conceptual diagram illustrating an example of time series data of the attitude angle obtained when the data acquisition device 21 is installed on the heel. FIG. 17 is a conceptual diagram illustrating an example of time series data of the attitude angle obtained when the data acquisition device 21 is installed on the toe. FIG. 18 is a conceptual diagram illustrating an example of time series data of the attitude angle obtained when the data acquisition device 21 is installed on the instep.

In the examples of FIGS. 16 to 18, after the minimal value (plantar flexion peak) of the attitude angle is detected, the maximal value (dorsiflexion peak) and the minimal value (plantar flexion peak) are alternately detected. A temporal change of the attitude angle becomes once gentle after the maximal value (the dorsiflexion peak) is detected, and then becomes large again. In the example in which the data acquisition device 21 is installed on the arch of the foot (FIG. 9), the maximal value is not detected in a period in which a temporal change of the attitude angle is gentle. On the other hand, in the examples of FIGS. 16 to 18, the maximal value is detected in a period in which a temporal change of the attitude angle becomes gentle.

When the time series data in FIGS. 16 to 18 is used as it is, after the maximal value of the dorsiflexion peak is detected, the minimal value and the maximal value that are not the plantar flexion peak and the dorsiflexion peak are detected. If the gait cycle of one step is determined based on the maximal value and the minimal value of the time series data of FIGS. 16 to 18, two steps are measured between the plantar flexion peak corresponding to the actual gait cycle of one step and the next plantar flexion peak, and an accurate gait cycle cannot be determined. Accordingly, in the examples of FIGS. 16 to 18, after the maximal value of the dorsiflexion peak is detected, an exclusion range for removing the minimal value and the maximal value that are not the plantar flexion peak and the dorsiflexion peak is set.

In the present example embodiment, the exclusion unit 224 of the gait cycle determination device 22 sets, as the exclusion range, a range from a value obtained by adding a second predetermined value a to the minimum value of the plantar flexion peak to a value obtained by subtracting a first predetermined value b from the maximum value of the dorsiflexion peak. For example, in a case where the data acquisition device 21 is installed on a high-heel shoe such as a high heel, the plantar surface is grounded in a state of being inclined with respect to the ground. If the exclusion range is set with reference to a predetermined center value, there is a possibility that the minimal value and the maximal value that are not a plantar flexion peak and a dorsiflexion peak deviate from the exclusion range in a case where the plantar surface is grounded in a state of being inclined with respect to the ground. Thus, in the present example embodiment, the exclusion range is set with reference to the minimum value of the plantar flexion peak and the maximum value of the dorsiflexion peak that are actually measured.

(Operation)

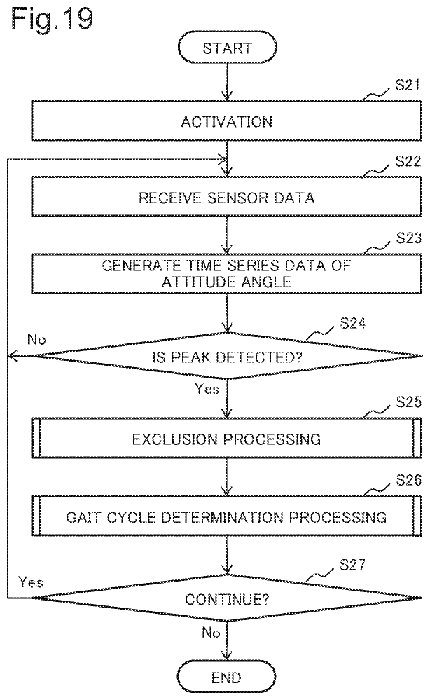

Next, operation of the gait cycle determination device 22 of the present example embodiment will be described with reference to the drawings. FIG. 19 is a flowchart for explaining the operation of the gait cycle determination device 22.

In FIG. 19, first, the gait cycle determination device 22 is activated (step S21).

Next, the gait cycle determination device 22 receives sensor data (acceleration data and angular velocity data) from the data acquisition device 21 (step S22).

Next, the gait cycle determination device 22 calculates the attitude angle using acceleration data and angular velocity data included in the received sensor data, and generates time series data of the attitude angle (step S23).

Then, in a case where a peak is detected (Yes in step S24), the gait cycle determination device 22 executes exclusion processing (step S25). On the other hand, in a case where no peak is detected (No in step S24), the processing returns to step S22.

Upon executing the exclusion processing (step S25), the gait cycle determination device 22 executes the gait cycle determination processing using the time series data of the attitude angle and outputs a determination result to the display device 23 (step S26). In the gait cycle determination processing, the gait cycle determination device 22 determines the gait cycle based on the order of the maximal peak and the minimal peak.

After step S26, in a case where the processing is continued (Yes in step S27), the processing returns to step S22. In a case where the processing is ended (No in step S27), the processing according to the flowchart of FIG. 19 is ended.

The example of the operation of the gait cycle determination device 22 has been described above. Note that the flowchart illustrated in FIG. 19 is an example, and the operation of the gait cycle determination device 22 of the present example embodiment is not limited to the procedure as it is.

[Exclusion Processing]

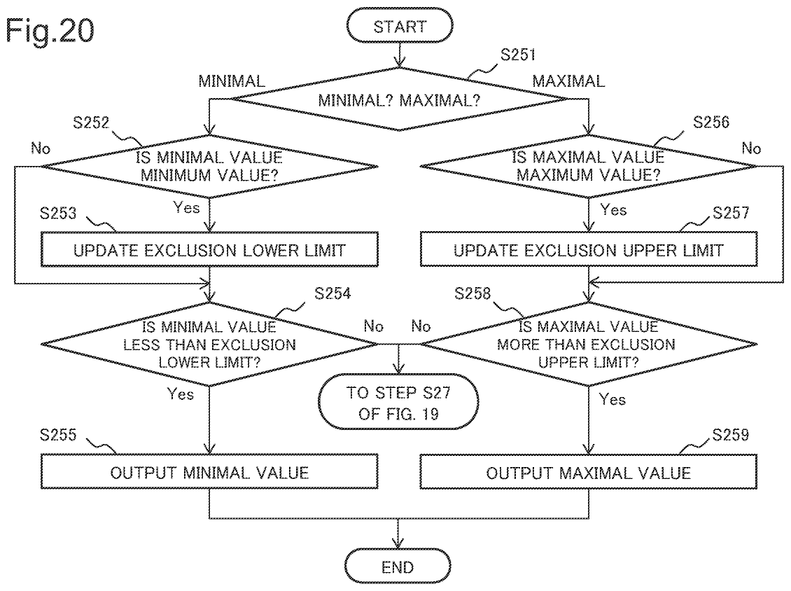

Next, exclusion processing by the exclusion unit 224 of the gait cycle determination device 22 of the present example embodiment will be described with reference to the drawings. FIG. 20 is a flowchart for explaining exclusion processing by the exclusion unit 224.

In FIG. 20, in a case where a minimal peak is received from the detection unit 222 (minimal in step S251), the exclusion unit 224 compares the minimal value with the minimal values received so far and determines whether the newly received minimal value is the minimum value (step S252).

In a case where the received minimal value is the minimum value (Yes in step S252), the exclusion unit 224 updates the exclusion lower limit (step S253). On the other hand, in a case where the received minimal value is not the minimum value (No in step S252), the exclusion unit 224 does not update the exclusion lower limit.

In a case where the received minimal value is less than the exclusion lower limit (Yes in step S254), the exclusion unit 224 outputs the minimal value to the determination unit 225 (step S255). After step S255, the processing proceeds to step S27 in the flowchart of FIG. 19. On the other hand, in a case where the received minimal value is equal to or more than the exclusion lower limit (No in step S254), the processing proceeds to step S27 in FIG. 19 without outputting the minimal value.

In FIG. 20, in a case where a maximal peak is received from the detection unit 222 (maximal in step S251), the exclusion unit 224 compares the maximal value with the maximal values received so far, and determines whether the newly received maximal value is the maximum value (step S256).

In a case where the received maximal value is the maximum value (Yes in step S256), the exclusion unit 224 updates the exclusion upper limit (step S257). On the other hand, in a case where the received maximal value is not the maximum value (No in step S256), the exclusion unit 224 does not update the exclusion upper limit.

In a case where the received maximal value is more than the exclusion upper limit (Yes in step S258), the exclusion unit 224 outputs the maximal value to the determination unit 225 (step S259). After step S259, the processing proceeds to step S26 in the flowchart of FIG. 19. On the other hand, in a case where the received maximal value is equal to or more than the exclusion upper limit (No in step S258), the processing proceeds to step S27 in FIG. 19 without outputting the maximal value.

The exclusion processing by the exclusion unit 224 has been described above. Note that the flowchart of FIG. 20 is an example, and the exclusion processing by the exclusion unit 224 of the present example embodiment is not limited to the procedure as it is.

[Gait Cycle Determination Processing]

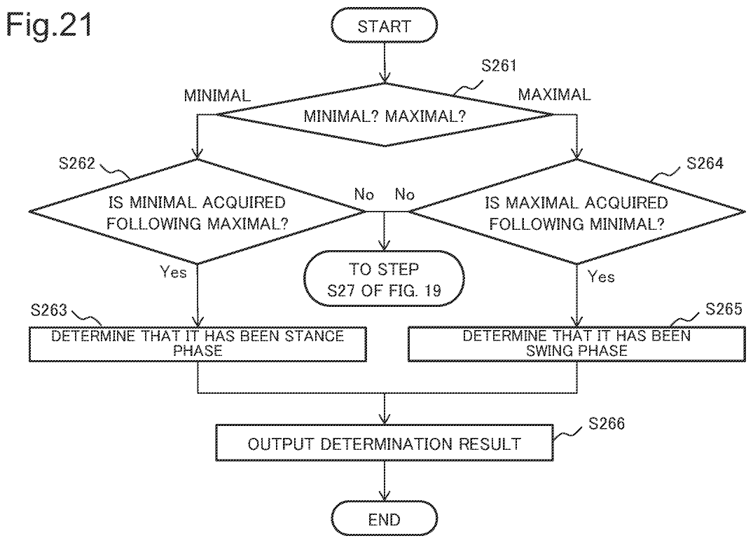

Next, the gait cycle determination processing by the determination unit 225 of the gait cycle determination device 22 of the present example embodiment will be described with reference to the drawings. FIG. 21 is a flowchart for explaining gait cycle determination processing by the determination unit 225.

In FIG. 21, in a case where the minimal value is acquired from the exclusion unit 224 (minimal in step S261), the determination unit 225 determines whether a minimal peak is acquired following a maximal peak (step S262). In a case where the minimal peak is acquired following the maximal peak (Yes in step S262), the determination unit 125 determines that the period before the minimal peak has been the stance phase (step S263) and outputs a determination result (step S266). In step S266, the determination unit 225 may output a determination result that the period before the minimal peak has been the stance phase, or may output a determination result that it is the swing phase at the current time. After step S266, the processing proceeds to step S27 in the flowchart of FIG. 19. On the other hand, in a case where the minimal peak is not acquired following the maximal peak (No in step S262), the processing proceeds to step S27 of the flowchart of FIG. 19.

On the other hand, in a case where the maximal value is acquired from the exclusion unit 224 (maximal in step S261), the determination unit 225 determines whether the maximal peak is acquired following the minimal peak (step S264). In a case where the maximal peak is acquired following the minimal peak (Yes in step S264), the determination unit 225 determines that the period before the maximal peak has been the swing phase (step S265), and outputs a determination result (step S266). In step S266, the determination unit 225 may output a determination result that the period before the maximal peak has been the swing phase, or may output a determination result that it is the stance phase at the current time. After step S266, the processing proceeds to step S27 in the flowchart of FIG. 19. On the other hand, in a case where the maximal peak is not acquired following the minimal peak (No in step S264), the processing proceeds to step S27 of the flowchart of FIG. 19.

The gait cycle determination processing by the determination unit 225 has been described above. Note that the flowchart of FIG. 21 is an example, and the gait cycle determination processing by the determination unit 225 of the present example embodiment is not limited to the procedure as it is.

As described above, the gait cycle determination system of the present example embodiment includes a first storage unit and an exclusion unit in addition to the reception unit, the detection unit, and the determination unit. The first storage unit stores at least a first predetermined value for setting an exclusion upper limit of the attitude angle and a second predetermined value for setting an exclusion lower limit of the attitude angle. The exclusion unit sets an exclusion range of the attitude angle based on the maximal value and the minimal value.

When the maximal value is received, the exclusion unit sets a value obtained by subtracting the first predetermined value from a maximum value of the maximal values that have been received previously as the exclusion upper limit, and outputs the maximal value that is received to the determination unit in a case where the maximal value is more than the exclusion upper limit. On the other hand, the exclusion unit does not output the maximal value that is received to the determination unit in a case where the maximal value is equal to or less than the exclusion upper limit.

When the minimal value is received, the exclusion unit sets a value obtained by adding the second predetermined value to a minimum value of the minimal values that have been received previously as the exclusion lower limit, and outputs the minimal value that is received to the determination unit in a case where the minimal value is less than the exclusion lower limit. On the other hand, the exclusion unit does not output the minimal value that is received to the determination unit in a case where the minimal value is equal to or more than the exclusion lower limit.

As one aspect of the present example embodiment, the first storage unit stores a maximum value of the maximal values that have been detected and a minimum value of the minimal values that have been detected. When the maximal value is received, in a case where the maximal value that is newly received is larger than the maximum value of the maximal values that have been detected and stored in the first storage unit, the exclusion unit updates the maximum value of the maximal values with the maximal value that is newly received. When the minimal value is received, in a case where the minimal value that is newly received is smaller than the minimum value of the minimal values that have been detected and stored in the first storage unit, the exclusion unit updates the minimum value of the minimal values with the minimal value that is newly received.

The gait cycle determination system of the present example embodiment excludes peak values that do not correspond to the timing at which the stance phase and the swing phase are switched and that are possible to occur due to the mounting position of the sensor. That is, with the gait cycle determination system of the present example embodiment, since the peak occurring at the timing at which the stance phase and the swing phase are switched can be excluded, the determination accuracy of the gait cycle is improved as compared with the first example embodiment.

### Third Example Embodiment

Next, a gait cycle determination system according to a third example embodiment of the present invention will be described with reference to the drawings. The gait cycle determination system of the present example embodiment is different from that of the first example embodiment in that the stance phase is subdivided and determined by defining a threshold that internally divides the period between a dorsiflexion peak and a plantar flexion peak at a predetermined ratio.

(Configuration)

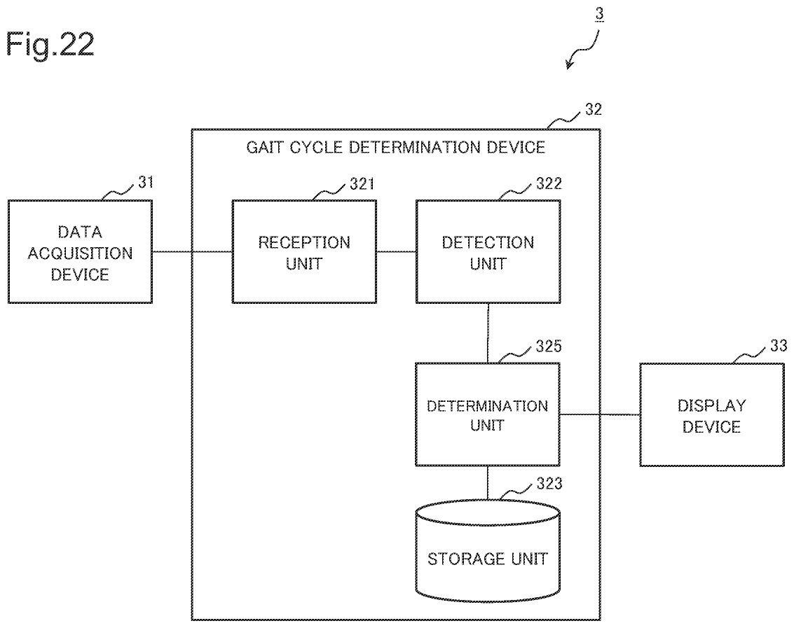

FIG. 22 is a block diagram schematically illustrating a configuration of the gait cycle determination system 3 of the present example embodiment. The gait cycle determination system 3 includes a data acquisition device 31, a gait cycle determination device 32, and a display device 33. The data acquisition device 31 and the gait cycle determination device 32 may be connected by wire or wirelessly. The gait cycle determination device 32 and the display device 33 may be connected by wire or wirelessly, or may be configured as the same terminal device. In a case where a determination result of the gait cycle determination device 32 is not displayed, the display device 33 may be deleted, and the data acquisition device 31 and the gait cycle determination device 32 may constitute the gait cycle determination system 3. Hereinafter, the data acquisition device 31 and the display device 33 are similar in configuration and function to the data acquisition device 11 and the display device 13, respectively, of the first example embodiment, and thus a detailed description thereof will be omitted.

As illustrated in FIG. 22, the gait cycle determination device 32 includes a reception unit 321, a detection unit 322, a storage unit 323, and a determination unit 325.

The reception unit 321 receives sensor data from the data acquisition device 31. The reception unit 321 outputs acceleration data and angular velocity data included in the sensor data to the detection unit 322.

The detection unit 322 acquires the acceleration data and the angular velocity data from the reception unit 321. The detection unit 322 calculates the attitude angle using the acquired acceleration data and angular velocity data, and generates time series data of the attitude angle. The detection unit 322 outputs the generated time series data of the attitude angle to the determination unit 325.

The detection unit 322 detects a maximal value and a minimal value from the time series data of the attitude angle. Upon detecting the maximal value from the time series data of the attitude angle, the detection unit 322 outputs the detected maximal value to the determination unit 325. Upon detecting the minimal value from the time series data of the attitude angle, the detection unit 322 outputs the detected minimal value to the determination unit 325. The maximal value and the minimal value output from the detection unit 322 include respective values of the maximal value and the minimal value and respective times when the maximal value and the minimal value is detected.

The storage unit 323 (also referred to as a second storage unit) stores a threshold (also referred to as a first threshold) of the attitude angle for determining a start time of the mid-stance period and a threshold (also referred to as a second threshold) of the attitude angle for determining a start time of a pre-swing period. A time at which the attitude angle coincides with the first threshold corresponds to the start time of the mid-stance period, and a time at which the attitude angle coincides with the second threshold corresponds to the start time of the pre-swing period.

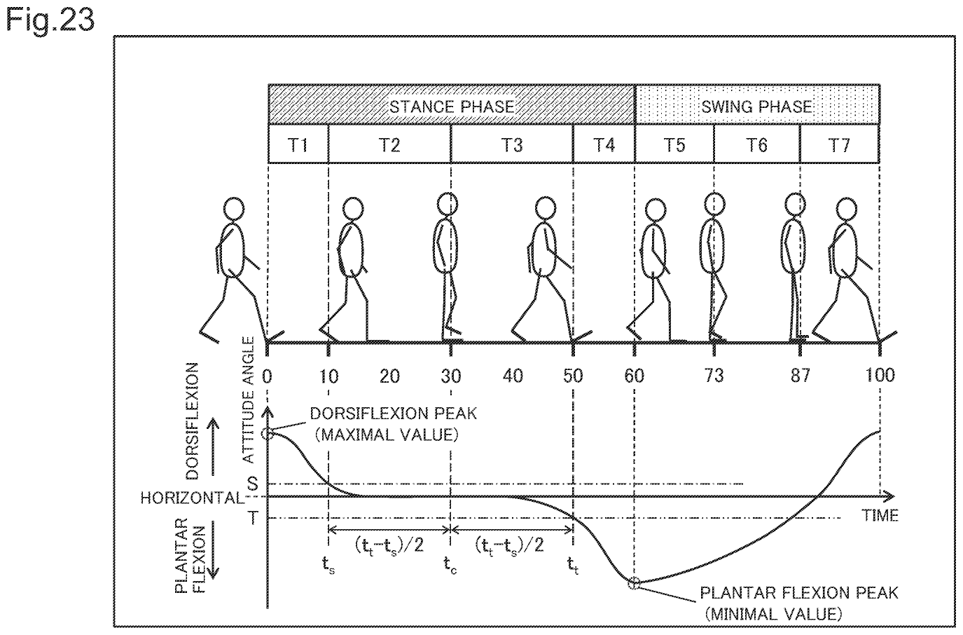

FIG. 23 is a conceptual diagram for explaining a gait cycle determined by the gait cycle determination device 32. The horizontal axis in FIG. 23 is a normalization time normalized by setting one gait cycle of one foot to 100 percent. In general, one gait cycle of one foot is roughly divided into a stance phase in which at least a part of the back side of the foot is in contact with the ground and a swing phase in which the back side of the foot is away from the ground. Further, the stance phase is divided into a loading response period T1, a mid-stance period T2, a terminal stance period T3, and a pre-swing period T4. The swing phase is divided into an initial swing period T5, a mid-swing period T6, and a terminal swing period T7.

A first threshold S and a second threshold T are preset thresholds. A time at which the attitude angle coincides with the first threshold S corresponds to a start time tₛ of the mid-stance period T2, and a time at which the attitude angle coincides with the second threshold corresponds to a start time tₜ of the pre-swing period T4. The first threshold and the second threshold are set based on a value obtained by internally dividing the dorsiflexion peak value (maximal value) and the plantar flexion peak value (minimal value) at a predetermined ratio. For example, the first threshold S and the second threshold T are only required to be configured such that the first threshold S and the second threshold T are set at a time of shipment based on an average value actually measured using a camera or a sensor, and adjusted for each user at a time of use.

An intermediate time between the start time tₛ of the mid-stance period T2 and the start time tₜ of the pre-swing period T4 corresponds to a start time t_{c} of the terminal stance period T3. The start time t_{c} of the terminal stance period T3 is calculated by following Equation 1.

t_{c}=(tₛ+tₜ)/2 (1)

The determination unit 325 acquires the minimal value or the minimal value from the detection unit 322. The determination unit 325 performs walking determination based on the order of acquiring the minimal value and the maximal value. In a case where the minimal value is acquired after acquiring the maximal value, the determination unit 325 determines that a transition from the stance phase to the swing phase has occurred. In a case where the maximal value is acquired after acquiring the minimal value, the determination unit 325 determines that a transition from the swing phase to the stance phase has occurred.

The determination unit 325 acquires the time series data of the attitude angle from the detection unit 322. The determination unit 325 subdivides the stance phase using the acquired time series data of the attitude angle. The determination unit 325 calculates a time at which the attitude angle matches the first threshold S as the start time tₛ of the mid-stance period T2, and calculates a time at which the attitude angle matches the second threshold T as the start time tₜ of the pre-swing period T4. The determination unit 325 calculates the start time t_{c} of the terminal stance period T3 using above Equation 1. The determination unit 325 subdivides the stance phase using the start time tₛ of the mid-stance period T2, the start time t_{c} of the terminal stance period T3, and the start time tₜ of the pre-swing period T4.

Specifically, the determination unit 325 determines a period from the dorsiflexion peak (maximal peak) to the start time tₛ of the mid-stance period T2 as the loading response period T1. The determination unit 325 determines a period from the start time tₛ of the mid-stance period T2 to the start time t_{c} of the terminal stance period T3 as the mid-stance period T2. The determination unit 325 determines a period from the start time t_{c} of the terminal stance period T3 to the start time tₜ of the pre-swing period T4 as the terminal stance period T3. The determination unit 325 determines a period from the start time tₜ of the pre-swing period T4 to the plantar flexion peak (minimal peak) as the pre-swing period T4.

The determination unit 325 outputs a determination result indicating whether it is the stance phase or the swing phase and a determination result obtained by subdividing the stance phase to the display device 33. In a case of a configuration not including the display device 33, the determination unit 325 outputs the determination result to a system or a device that is not illustrated.

The example of the configuration of the gait cycle determination device 32 has been described above. Note that the configuration of FIG. 22 is an example, and the configuration of the gait cycle determination device 32 included in the gait cycle determination system 3 of the present example embodiment is not limited to the mode as it is. The gait cycle determination device 32 may be replaced with the gait cycle determination device 22 of the gait cycle determination system 2 of the second example embodiment.

(Operation)

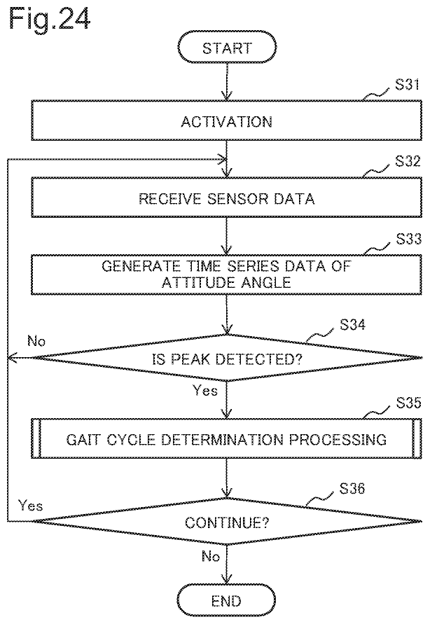

Next, operation of the gait cycle determination device 32 of the present example embodiment will be described with reference to the drawings. FIG. 24 is a flowchart for explaining the operation of the gait cycle determination device 32.

In FIG. 24, first, the gait cycle determination device 32 is activated (step S31).

Next, the gait cycle determination device 32 receives sensor data (acceleration and angular velocity) from the data acquisition device 31 (step S32).

Next, the gait cycle determination device 32 calculates the attitude angle using acceleration data and angular velocity data included in the received sensor data, and generates time series data of the attitude angle (step S33).

Then, in a case where a peak is detected (Yes in step S34), the gait cycle determination device 32 executes the gait cycle determination processing (step S35) using the time series data of the attitude angle, and outputs a determination result to the display device 33. In the gait cycle determination processing (step S35), the gait cycle determination device 32 determines the gait cycle based on the order of the maximal peak and the minimal peak. On the other hand, in a case where no peak is detected (No in step S34), the processing returns to step S32.

After step S35, in a case where the processing is continued (Yes in step S36), the processing returns to step S32. In a case where the pointing processing is ended (No in step S36), the processing according to the flowchart of FIG. 24 ends.

The example of the operation of the gait cycle determination device 32 has been described above. Note that the flowchart of FIG. 24 is an example, and the operation of the gait cycle determination device 32 of the present example embodiment is not limited to the procedure as it is.

[Gait Cycle Determination Processing]

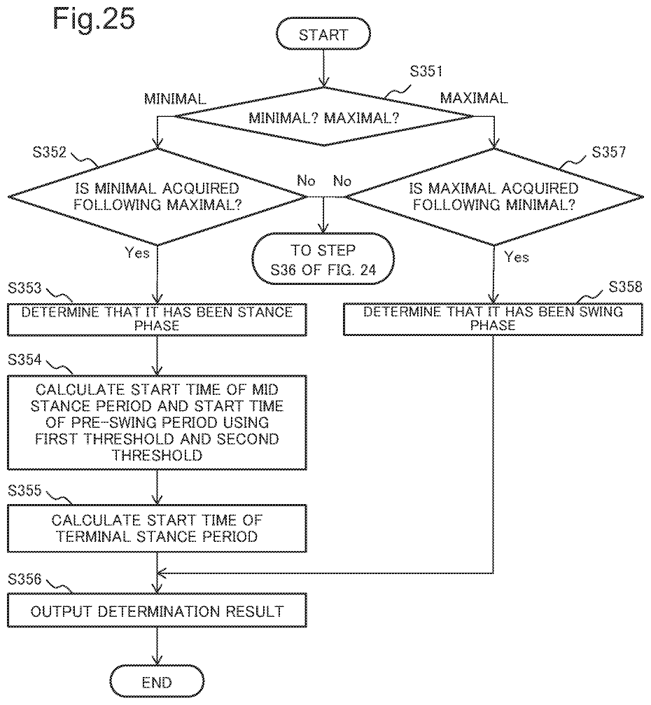

Next, the gait cycle determination processing by the determination unit 325 of the gait cycle determination device 32 of the present example embodiment will be described with reference to the drawings. FIG. 25 is a flowchart for explaining gait cycle determination processing by the determination unit 325.

In FIG. 25, in a case where a minimal peak is acquired (minimal in step S351), the determination unit 325 determines whether a minimal peak is acquired following the maximal peak (step S352). In a case where a minimal peak is acquired following the maximal peak (Yes in step S352), the determination unit 325 determines that the period before the minimal peak has been the stance phase (step S353). On the other hand, in a case where the minimal peak is not acquired following the maximal peak (No in step S352), the processing proceeds to step S36 of the flowchart of FIG. 24.

Upon determining that the period before the minimal peak has been the stance phase (step S353), the determination unit 325 calculates the start time of the mid-stance period and the start time of the pre-swing period using the first threshold and the second threshold (step S354).

Next, the determination unit 325 calculates the start time of the terminal stance period using the start time of the mid-stance period and the start time of the pre-swing period (step S355).

Then, the determination unit 325 outputs a determination result (step S356). In step S356, the determination unit 325 may output a determination result that the period before the minimal peak has been the stance phase, or may output a determination result that it is the swing phase at the current time. The determination unit 325 outputs the start time of the mid-stance period, the start time of the terminal stance period, and the start time of the pre-swing period as determination results. After step S356, the processing proceeds to step S36 in the flowchart of FIG. 24.

In FIG. 25, in a case where the maximal peak is acquired (maximal in step S351), the determination unit 325 determines whether the maximal peak is acquired following the minimal peak (step S357). In a case where the maximal peak is acquired following the minimal peak (Yes in step S357), the determination unit 325 determines that the period before the maximal peak has been the swing phase (step S358), and outputs the determination result (step S356). In step S356, the determination unit 325 may output a determination result that the period before the maximal peak has been the swing phase, or may output a determination result that it is the stance phase at the current time. After step S356, the processing proceeds to step S36 in the flowchart of FIG. 24. On the other hand, in a case where the maximal peak is not acquired following the minimal peak (No in step S357), the processing proceeds to step S36 of the flowchart of FIG. 24.

The gait cycle determination processing by the determination unit 325 has been described above. Note that the flowchart of FIG. 25 is an example, and the gait cycle determination processing by the determination unit 325 of the present example embodiment is not limited to the procedure as it is.

As described above, the gait cycle determination system of the present example embodiment includes a second storage unit in addition to the reception unit, the detection unit, and the determination unit. The second storage unit stores at least a first threshold of the attitude angle for determining a start time of a mid-stance period and a second threshold of the attitude angle for determining a start time of a pre-swing period. The determination unit calculates a time at which the attitude angle matches the first threshold as the start time of the mid-stance period, and calculates a time at which the attitude angle matches the second threshold as the start time of the pre-swing period. Then, the determination unit calculates an intermediate time between the start time of the mid-stance period and the start time of the pre-swing period as a start time of a terminal stance period.

The gait cycle determination system of the present example embodiment subdivides and determines the stance period. Thus, with the gait cycle determination system of the present example embodiment, more advanced walk analysis than in the first example embodiment is possible.

### Fourth Example Embodiment

Next, a gait cycle determination system according to a fourth example embodiment of the present invention will be described with reference to the drawings. The gait cycle determination system of the present example embodiment calculates the attitude angle by using sensor data acquired by an acceleration sensor and an angular velocity sensor disposed on both left and right footwear. The gait cycle determination system of the present example embodiment is different from that of the first example embodiment in that the gait cycle is determined based on time series data of attitude angles of both the right and left feet.

(Configuration)

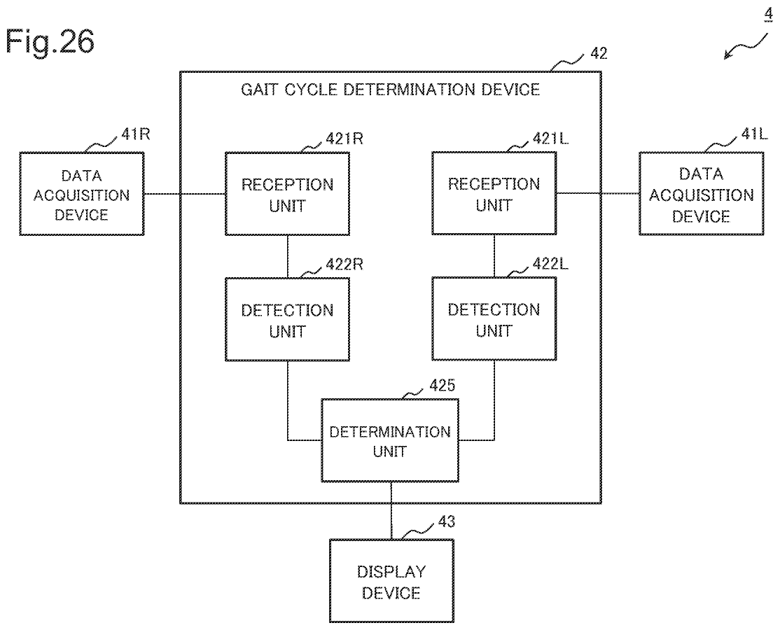

FIG. 26 is a block diagram schematically illustrating a configuration of the gait cycle determination system 4 of the present example embodiment. The gait cycle determination system 4 includes a data acquisition device 41R, a data acquisition device 41L, a gait cycle determination device 42, and a display device 43. The data acquisition device 41R and the data acquisition device 41L have similar configurations and functions. Each of the data acquisition device 41R and the data acquisition device 41L and the gait cycle determination device 42 may be connected by wire or wirelessly. The gait cycle determination device 42 and the display device 43 may be connected by wire or wirelessly, or may be configured as the same terminal device. In a case where the determination result of the gait cycle determination device 42 is not displayed, the display device 43 may be deleted, and the data acquisition device 41R, the data acquisition device 41L, and the gait cycle determination device 42 may constitute the gait cycle determination system 4. Hereinafter, the data acquisition device 41R and the data acquisition device 41L are similar in configuration and function to the data acquisition device 11 of the first example embodiment, and the display device 23 is similar in configuration and function to the display device 13 of the first example embodiment, and thus detailed description thereof is omitted.

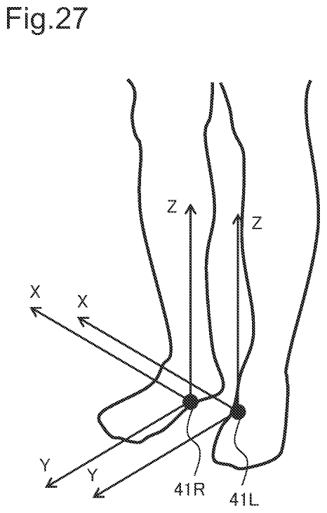

FIG. 27 is a conceptual diagram for explaining a coordinate system of sensor data acquired by each of the data acquisition device 41R and the data acquisition device 41L. In the example of FIG. 27, the lateral direction of the walker is set to the X-axis direction (rightward direction is positive), the traveling direction of the walker is set to the Y-axis direction (forward direction is positive), and the gravity direction is set to the Z-axis direction (vertically upward direction is positive). In the present example embodiment, an example in which sensor data acquired by the data acquisition device 41R arranged on footwear for a right foot is mainly used will be described. In practice, in this configuration, the sensor data acquired by the data acquisition device 41L arranged on footwear for a left foot may be mainly used, or the sensor data acquired by both the data acquisition device 41R and the data acquisition device 41L may be mainly used.

The data acquisition device 41R (also referred to as a first sensor) is placed on footwear for a right foot of a user. The data acquisition device 41R converts data acquired by the acceleration sensor and the angular velocity sensor into digital data (sensor data), and transmits sensor data after conversion to the gait cycle determination device 42.

The data acquisition device 41L (also referred to as a second sensor) is placed on footwear for a left foot of the user. The data acquisition device 41L converts data acquired by the acceleration sensor and the angular velocity sensor into digital data (sensor data), and transmits the sensor data after conversion to the gait cycle determination device 42.

As illustrated in FIG. 26, the gait cycle determination device 42 includes a reception unit 421R, a reception unit 421L, a detection unit 422R, a detection unit 422L, and a determination unit 425.

The reception unit 421R (also referred to as a first reception unit) receives the sensor data of the right foot from the data acquisition device 41R disposed on the footwear on the right foot side (also referred to as first footwear). The reception unit 421R outputs acceleration data and angular velocity data included in the sensor data of the right foot to the detection unit 422R.

The detection unit 422R (also referred to as a first detection unit) acquires the acceleration data and the angular velocity data of the right foot from the reception unit 421R. The detection unit 422R calculates the attitude angle of the right foot using the acquired acceleration data and angular velocity data, and generates time series data of the attitude angle of the right foot. The detection unit 422R detects a maximal value or a minimal value from the time series data of the attitude angle of the right foot. Upon detecting the maximal value from the time series data of the attitude angle of the right foot, the detection unit 422R outputs the detected maximal value to the determination unit 425. Upon detecting the minimal value from the time series data of the attitude angle of the right foot, the detection unit 422R outputs the detected minimal value to the determination unit 425. Each of the maximal value and the minimal value output from the detection unit 422R includes a value of each of the maximal value and the minimal value and a time when each of the maximal value and the minimal value is detected.

The reception unit 421L (also referred to as a second reception unit) receives sensor data of the left foot from the data acquisition device 41L arranged on the footwear on the left foot side (also referred to as second footwear). The reception unit 421L outputs acceleration data and angular velocity data included in the sensor data of the left foot to the detection unit 422L.

The detection unit 422L (also referred to as a second detection unit) acquires the acceleration data and the angular velocity data of the left foot from the reception unit 421L. The detection unit 422L calculates the attitude angle of the left foot using the acquired acceleration data and angular velocity data, and generates time series data of the attitude angle of the left foot. The detection unit 422L detects a maximal value or a minimal value from the time series data of the attitude angle of the left foot. Upon detecting the maximal value from the time series data of the attitude angle of the left foot, the detection unit 422L outputs the detected maximal value to the determination unit 425. Upon detecting the minimal value from the time series data of the attitude angle of the left foot, the detection unit 422L outputs the detected minimal value to the determination unit 425. Each of the maximal value and the minimal value output from the detection unit 422L includes a value of each of the maximal value and the minimal value and a time when each of the maximal value and the minimal value is detected.

The determination unit 425 acquires the minimal value and the minimal value from each of the detection unit 422R and the detection unit 422L. The determination unit 425 performs walking determination based on an order of acquiring the minimal value and the maximal value.

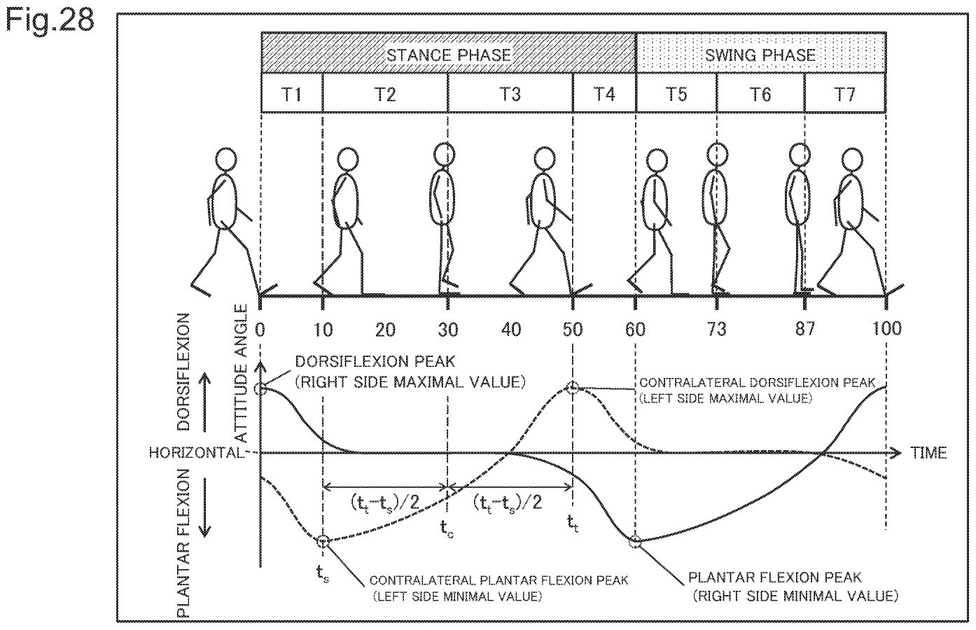

FIG. 28 is a conceptual diagram for explaining the gait cycle determined by the gait cycle determination device 42. The horizontal axis in FIG. 28 is a normalization time normalized by setting one gait cycle of the right foot to 100 percent. In general, one gait cycle of one foot is roughly divided into a stance phase in which at least a part of the back side of the foot is in contact with the ground and a swing phase in which the back side of the foot is away from the ground. Further, the stance phase is divided into a loading response period T1, a mid-stance period T2, a terminal stance period T3, and a pre-swing period T4. The swing phase is divided into an initial swing period T5, a mid-swing period T6, and a terminal swing period T7. FIG. 28 illustrates a change (broken line) in the attitude angle in one gait cycle of the left foot in correspondence with a change (solid line) in the attitude angle in one gait cycle of the right foot.

The determination unit 425 determines a time when the attitude angle of one foot (right foot) becomes maximal (dorsiflexion peak) as a start time of the stance phase, and determines a time when the attitude angle of the one foot (right foot) becomes minimal (plantar flexion peak) as a start time of the swing phase. The determination unit 425 determines a time when the attitude angle of the contralateral foot (left foot) becomes minimal (contralateral plantar flexion peak) as a start time of the mid-stance period T2, and determines a time when the attitude angle of the contralateral foot (left foot) becomes maximal (contralateral dorsiflexion peak) as a start time of the pre-swing period T4.

The determination unit 425 determines the gait cycle of one foot (right foot) based on an order relationship between a dorsiflexion peak at which the attitude angle of the one foot (right foot) becomes maximal and a plantar flexion peak at which the attitude angle of the one foot (right foot) becomes minimal. The determination unit 425 determines a period from the dorsiflexion peak (maximal) to the next plantar flexion peak (minimal) of one foot (right foot) as the stance phase of the one foot (right foot), and a period from the plantar flexion peak (minimal) to the next dorsiflexion peak (maximal) of one foot (right foot) as the swing phase of the one foot (right foot). That is, in a case where the minimal value is detected after the maximal value with respect to one foot (right foot), the determination unit 425 determines that a transition from the stance phase to the swing phase has occurred. On the other hand, in a case where the maximal value is detected after the minimal value with respect to one foot (right foot), the determination unit 425 determines that a transition from the swing phase to the stance phase has occurred.

Further, the determination unit 425 determines the gait cycle of one foot (right foot) including an order relationship between the contralateral plantar flexion peak at which the attitude angle of the contralateral foot (left foot) becomes minimal and the contralateral dorsiflexion peak at which the attitude angle of the contralateral foot (left foot) becomes maximal. The determination unit 425 determines a time of the contralateral plantar flexion peak at which the attitude angle of the contralateral foot (left foot) becomes minimal as the start time tₛ of the mid-stance period T2 of one foot (right foot). The determination unit 425 determines a time of the contralateral dorsiflexion peak at which the attitude angle of the contralateral foot (left foot) becomes maximal as the start time tₜ of the pre-swing period T4 of one foot (right foot). The determination unit 425 calculates the start time t_{c} of the terminal stance period T3 using Equation 1 of the third example embodiment. The determination unit 425 subdivides the stance phase using the start time tₛ of the mid-stance period T2, the start time t_{c} of the terminal stance period T3, and the start time tₜ of the pre-swing period T4.

Specifically, the determination unit 425 determines a period from the dorsiflexion peak (maximal peak) to the start time tₛ of the mid-stance period T2 as the loading response period T1. The determination unit 425 determines a period from the start time tₛ of the mid-stance period T2 to the start time t_{c} of the terminal stance period T3 as the mid-stance period T2. The determination unit 425 determines a period from the start time t_{c} of the terminal stance period T3 to the start time tₜ of the pre-swing period T4 as the terminal stance period T3. The determination unit 425 determines a period from the start time tₜ of the pre-swing period T4 to the plantar flexion peak (minimal peak) as the pre-swing period T4.

The determination unit 425 outputs a determination result indicating whether it is the stance phase or the swing phase and a determination result obtained by subdividing the stance phase to the display device 43. In a case of a configuration not including the display device 43, the determination unit 425 outputs the determination result to a system or a device that is not illustrated.

The example of the configuration of the gait cycle determination device 42 has been described above. Note that the configuration of FIG. 26 is an example, and the configuration of the gait cycle determination device 42 included in the gait cycle determination system 4 of the present example embodiment is not limited to the mode as it is. Some of the functions of the exclusion unit 224 of the second example embodiment and the determination unit 325 of the third example embodiment may be added to the gait cycle determination device 42.

As described above, the gait cycle determination system of the present example embodiment includes a reception unit including a first reception unit and a second reception unit, a detection unit including a first detection unit and a second detection unit, and a determination unit. The first reception unit receives the sensor data acquired by a first sensor installed on first footwear. The second reception unit receives the sensor data acquired by a second sensor installed on second footwear. The first detection unit generates time series data of the attitude angle of a first foot by using acceleration and angular velocity included in the sensor data received by the first reception unit, and detects the maximal value and the minimal value from the time series data of the attitude angle of the first foot. The second detection unit generates time series data of the attitude angle of a second foot by using acceleration and angular velocity included in the sensor data received by the second reception unit, and detects the maximal value and the minimal value from the time series data of the attitude angle of the second foot. The determination unit determines a detection time of the minimal value detected from time series data of the attitude angle of the second foot as a start time of a mid-stance period of the first foot, and determines a detection time of the maximal value detected from time series data of the attitude angle of the second foot as a start time of a swing of the first foot. Then, the determination unit determines an intermediate time between the detection time of the minimal value and the detection time of the maximal value detected from the time series data of the attitude angle of the second foot as a start time of a terminal stance period of the first foot.

The gait cycle determination system of the present example embodiment generates the time series data of the attitude angle for each of the left and right foot portions. The gait cycle determination system of the present example embodiment determines the gait cycle of one foot portion (first foot) based on the maximal value and the minimal value of the time series data of the attitude angle of the one foot portion (first foot). The gait cycle determination system of the present example embodiment determines the stance phase of one foot portion (first foot) by subdividing the stance phase based on the maximal value and the minimal value of the time series data of the attitude angle of the other foot portion (second foot). Thus, with the gait cycle determination system of the present example embodiment, more advanced walk analysis than in the first example embodiment is possible. With the gait cycle determination system of the present example embodiment, the stance phase is subdivided and determined based on the actual measurement value, and thus it is possible to perform the walk analysis with higher accuracy than the gait cycle determination system of the third example embodiment.

(Hardware)

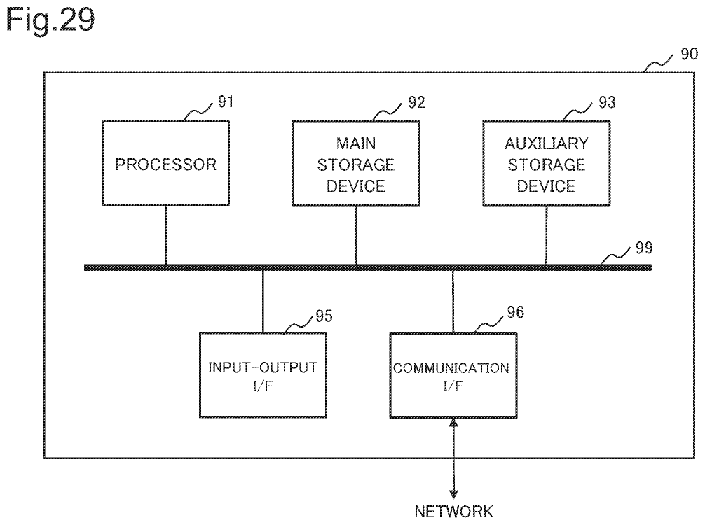

Here, a hardware configuration that executes the processing of the gait cycle determination device according to each example embodiment of the present invention will be described using the information processing device 90 of FIG. 29 as an example. Note that the information processing device 90 in FIG. 29 is a configuration example for executing the processing of the gait cycle determination device of each example embodiment, and does not limit the scope of the present invention.

As illustrated in FIG. 29, the information processing device 90 includes a processor 91, a main storage device 92, an auxiliary storage device 93, an input-output interface 95, and a communication interface 96. In FIG. 29, the interface is abbreviated as I/F. The processor 91, the main storage device 92, the auxiliary storage device 93, the input-output interface 95, and the communication interface 96 are data-communicably connected to each other via a bus 99. The processor 91, the main storage device 92, the auxiliary storage device 93, and the input-output interface 95 are connected to a network such as the Internet or an intranet via the communication interface 96.

The processor 91 develops a program stored in the auxiliary storage device 93 or the like in the main storage device 92 and executes the developed program. In the present example embodiment, it is only required to use a software program installed in the information processing device 90. The processor 91 executes processing by the gait cycle determination device according to the present example embodiment.

The main storage device 92 has an area in which a program is developed. The main storage device 92 is only required to be, for example, a volatile memory such as a dynamic random access memory (DRAM). A nonvolatile memory such as a magnetoresistive random access memory (MRAM) may be configured and added as the main storage device 92.

The auxiliary storage device 93 stores various data. The auxiliary storage device 93 includes a local disk such as a hard disk or a flash memory. In addition, the main storage device 92 may be configured to store various data, and the auxiliary storage device 93 may be omitted.

The input-output interface 95 is an interface for connecting the information processing device 90 and a peripheral device. The communication interface 96 is an interface for connecting to an external system or device through a network such as the Internet or an intranet based on a standard or a specification. The input-output interface 95 and the communication interface 96 may be shared as an interface connected to an external device.

An input device such as a keyboard, a mouse, or a touch panel may be connected to the information processing device 90 as necessary. These input devices are used to input information and settings. In a case where the touch panel is used as the input device, the display screen of the display device is only required to also serve as the interface of the input device. Data communication between the processor 91 and the input device is only required to be mediated by the input-output interface 95.

The information processing device 90 may be provided with a display device for displaying information. In a case where a display device is provided, the information processing device 90 preferably includes a display control device (not illustrated) for controlling display of the display device. The display device is only required to be connected to the information processing device 90 via the input-output interface 95.

The information processing device 90 may be provided with a disk drive as necessary. The disk drive is connected to the bus 99. The disk drive mediates reading of data and/or a program from a storage medium, writing of a processing result of the information processing device 90 to the storage medium, and the like between the processor 91 and the storage medium (program storage medium), which is not illustrated. The storage medium can be achieved by, for example, an optical storage medium such as a compact disc (CD) or a digital versatile disc (DVD). The storage medium may be achieved by a semiconductor storage medium such as a universal serial bus (USB) memory or a secure digital (SD) card, a magnetic storage medium such as a flexible disk, or another storage medium.

The above is an example of a hardware configuration for enabling the gait cycle determination device according to each example embodiment of the present invention. Note that the hardware configuration of FIG. 29 is an example of a hardware configuration for executing the processing of the gait cycle determination device according to each example embodiment, and does not limit the scope of the present invention. A program for causing a computer to execute processing related to the gait cycle determination device according to each example embodiment is also included in the scope of the present invention. Further, a program storage medium in which the program according to each example embodiment is stored is also included in the scope of the present invention.

The components of the gait cycle determination device of each example embodiment can be freely combined. The components of the gait cycle determination device of each example embodiment may be achieved by software or may be achieved by a circuit.

While the present invention has been particularly shown and described with reference to example embodiments thereof, the invention is not limited to these example embodiments. It will be understood by those of ordinary skill in the art that various changes in form and details may be made therein without departing from the spirit and scope of the present invention as defined by the claims.

### REFERENCE SIGNS LIST

1, 2, 3, 4 gait cycle determination system
11, 21, 31, 41R, 41L data acquisition device
12, 22, 32, 42 gait cycle determination device
13, 23, 33, 43 display device
111 acceleration sensor
112 angular velocity sensor
113 signal processing unit
114 data transmission unit
121, 221, 321 reception unit
122, 222, 322 detection unit
125, 225, 325 determination unit
223, 323 storage unit
224 exclusion unit
421R, 421L reception unit
422R, 422L detection unit
425 determination unit

What is claimed is:

1. A gait cycle determination system comprising:
a receiver configured to receive sensor data including acceleration and angular velocity acquired by a sensor installed on footwear;
at least one memory storing instructions; and
at least one processor connected to the at least one memory and configured to execute the instructions to:
generate time series data of an attitude angle of at least one foot by using the acceleration and the angular velocity included in the sensor data,
detect a maximal value and a minimal value from the time series data of the attitude angle; and
determine a gait cycle based on an order of the maximal value and the minimal value.

2. The gait cycle determination system according to claim 1, wherein
the at least one processor is configured to execute the instructions to
determine a gait phase in a period from a detection time of the maximal value to a detection time of the minimal value that is next as a stance phase, and
determine the gait phase in a period from a detection time of the minimal value to a detection time of the maximal value that is next as a swing phase.

3. The gait cycle determination system according to claim 1, further comprising:
a first storage configured to store at least a first predetermined value for setting an exclusion upper limit of the attitude angle and a second predetermined value for setting an exclusion lower limit of the attitude angle, wherein
the at least one processor is configured to execute the instructions to
set an exclusion range of the attitude angle based on the maximal value and the minimal value,
set, in response to a receipt of the maximal value, a value obtained by subtracting the first predetermined value from a maximum value of the maximal values that have been received previously as the exclusion upper limit, determine the gait cycle by using the maximal value that is received in a case where the maximal value is more than the exclusion upper limit, and do not determine the gait cycle by using the maximal value that is received in a case where the maximal value is equal to or less than the exclusion upper limit, and
set, in response to a receipt of the minimal value, a value obtained by adding the second predetermined value to a minimum value of the minimal values that have been received previously as the exclusion lower limit, determine the gait cycle by using the minimal value that is received in a case where the minimal value is less than the exclusion lower limit, and do not determine the gait cycle by using the minimal value that is received in a case where the minimal value is equal to or more than the exclusion lower limit.

4. The gait cycle determination system according to claim 3, wherein
the first storage is configured to
store a maximum value of the maximal values that have been detected and a minimum value of the minimal values that have been detected are stored, and
the at least one processor is configured to execute the instructions to
update, in response to a receipt of the maximal value, in a case where the maximal value that is newly received is larger than the maximum value of the maximal values that have been detected and stored in the first storage, the maximum value of the maximal values with the maximal value that is newly received, and
update, in response to a receipt of the minimal value, in a case where the minimal value that is newly received is smaller than the minimum value of the minimal values that have been detected and stored in the first storage, the minimum value of the minimal values with the minimal value that is newly received.

5. The gait cycle determination system according to claim 1, further comprising
a second storage configured to store at least a first threshold of the attitude angle for determining a start time of a mid-stance period and a second threshold of the attitude angle for determining a start time of a pre-swing period, wherein
the at least one processor is configured to execute the instructions to
calculate a time at which the attitude angle matches the first threshold as the start time of the mid-stance period,
calculate a time at which the attitude angle matches the second threshold as the start time of the pre-swing period, and
calculate an intermediate time between the start time of the mid-stance period and the start time of the pre-swing period as a start time of a terminal stance period.

6. The gait cycle determination system according to claim 1, wherein
the receiver includes:
a first receiver configured to receive the sensor data acquired by a first sensor installed on first footwear; and
a second receiver configured to receive the sensor data acquired by a second sensor installed on second footwear, wherein
the at least one processor is configured to execute the instructions to
generate time series data of the attitude angle of a first foot by using acceleration and angular velocity included in the sensor data received by the first receiver,
detect the maximal value and the minimal value from the time series data of the attitude angle of the first foot;
generate time series data of the attitude angle of a second foot by using acceleration and angular velocity included in the sensor data received by the second receiver,
detect the maximal value and the minimal value from the time series data of the attitude angle of the second foot,
determine a detection time of the minimal value detected from time series data of the attitude angle of the second foot as a start time of a mid-stance period of the first foot,
determine a detection time of the maximal value detected from time series data of the attitude angle of the second foot as a start time of a pre-swing period of the first foot, and
determine an intermediate time between the detection time of the minimal value and the detection time of the maximal value detected from the time series data of the attitude angle of the second foot as a start time of a terminal stance period of the first foot.

7. The gait cycle determination system according to claim 1, further comprising a data acquisition device that is installed on footwear, detects the acceleration and the angular velocity, generates the sensor data including the acceleration and the angular velocity that are detected, and transmits the sensor data that is generated to the receiver.

8. The gait cycle determination system according to claim 1, further comprising a display device that acquires a determination result by the determination and displays the acquired determination result.

9. A gait cycle determination method comprising:
receiving sensor data including acceleration and angular velocity acquired by a sensor installed on at least one footwear;
generating time series data of an attitude angle of at least one foot by using the acceleration and the angular velocity included in the sensor data;
detecting a maximal value and a minimal value from the time series data of the attitude angle; and
determining a gait cycle based on an order of the maximal value and the minimal value.

10. A non-transient program storage medium storing a program that causes a computer to execute:
a process of receiving sensor data including acceleration and angular velocity acquired by a sensor installed on at least one footwear;
a process of generating time series data of an attitude angle of at least one foot by using the acceleration and the angular velocity included in the sensor data;
a process of detecting a maximal value and a minimal value from the time series data of the attitude angle; and
a process of determining a gait cycle based on an order of the maximal value and the minimal value.
